(19) United States
(12) Patent Application Publication
(10) Pub. No.: US 2025/0231187 A1
(43) Pub. Date: Jul. 17, 2025

(54) HERPES SIMPLEX VIRUS TYPES 1 AND 2 LATERAL FLOW ASSAY

(71) Applicant: VIRO RESEARCH, LLC, Snellville, GA (US)

(72) Inventors: Nicholas VAFAI, Snellville, GA (US); Dr. Abbas VAFAI, Snellville, GA (US)

(21) Appl. No.: 18/699,017

(22) PCT Filed: Oct. 5, 2022
(86) PCT No.: PCT/US2022/077593

§ 371 Date: Apr. 5, 2024

Related U.S. Application Data
(60) Provisional application No. 63/252,414, filed on Oct. 5, 2021.

(51) Int. Cl. G01N 33/569; C07K 16/08; G01N 33/543

(52) U.S. Cl. CPC G01N 33/56994; C07K 16/087; G01N 33/54388; G01N 2333/035; G01N 2469/20

(57) ABSTRACT

Generally, the present disclosure is directed to an antibody detection lateral flow assay that is specific to antibodies binding a portion of the gG protein expressed by HSV type 1 and/or type 2. The assay can be based on visual labels and interpreted without the use of a reader to provide for quick and easy detection. Multiple colored labels can be used for evaluation, and these colored labels can be covalently attached to the portion of the gG protein and the resulting modified gG-label complex applied to a substrate (e.g., backing card) for use in a binding assay.

### FIELD

The present application is directed to systems, methods, and apparatuses for antibody detection. In particular, the present disclosure presents an example lateral flow assay for detection of antibodies in human serum, whole blood and finger stick against Herpes Simplex type I and II Virus (HSV-1 and HSV-2), and more particularly, an assay incorporating an isolated peptide fragment of structural glycoprotein gG from HSV-1 and/or HSV-2.

### BACKGROUND

HSV-1 and HSV-2 are a members of the alpha herpes virus family. About 1 out of 6 adults in the United States have antibodies to HSV-2, the virus typically linked to genital herpes.

A herpes infection cannot be cured. After becoming infected with HSV, the virus stays in the body for life. The virus “hides” in a certain type of nerve cell and causes more outbreaks of sores in some people. Recurring infections can be triggered by stress, fatigue, sunlight, or another infection, such as a cold or flu. Medicine can relieve symptoms and shorten the length of the outbreaks, but medicine cannot cure the infection.

Tests for HSV are most often done only for sores in the genital area. The test may also be done using other types of samples, such as spinal fluid, blood, urine, or tears. To see whether sores are caused by HSV, different types of tests may be done.

HSV DNA encodes multiple glycoproteins that can be found in infected cells. These glycoproteins can produce immunogenic responses that elicit neutralizing antibody secretion. Current HSV antibody detection systems utilize viral lysate as part of an ELISA capture system; however, these capture assays suffer several drawbacks including long read time, waste from excess reagents, and inability to work with typical patient samples such as whole blood.

There remains a need for improved HSV antibody detection systems that can be designed for mass production via incorporation of expressed isolated peptides, and which can be used on patient samples, for example, in point-of-care or home applications. Further, such systems may be most beneficial if results can be validated within a short timeframe (e.g., within 1 hour).

### SUMMARY

In one embodiment, the present application is directed to an antibody detection lateral flow assay that is specific to antibodies binding a portion of the gG protein(s) expressed by HSV-1 and/or HSV-2 (for referencing purposes, the portion of the gG protein expressed by HSV-1 is referred to as G1 sequence and the portion of the gG protein expressed by HSV-2 is referred to as G2 sequence, hereafter).

In some aspects of this embodiment, the assay is based on visual labels, and interpreted without the use of a reader, to provide for quick and easy detection. In other embodiments, the assay is quantitative, and is designed for use with a reader system. In some embodiments, the assay is a sandwich assay, and in other embodiments, the assay is a competitive assay.

One or more colored labels can be used for evaluation, such as colloidal gold, colored latex, cellulose nanobeads, fluorescent proteins, etc. These colored labels can be chemically or physically attached (for example, covalently bonded, ionically bonded, bonded by chemisorption or physisorption, bonded via functional linking groups or linker molecules, or bonded via hydrogen bonding, hydrophobic interaction, or Van Der Waals attraction), attached to the portion of the isolated G1 and/or G2 proteins and the resulting modified G1/G2-label complex applied to a substrate (e.g., backing card) for use in a binding assay.

Further, the G1 and/or G2 proteins (also referred to herein as an isolated G1 and/or G2 peptide) can be isolated via an expression system, rather than a viral lysate, to produce a more uniform test composition that would likely display improved reproducibility relative to ELISA assays.

The assay involves testing a biological sample. The biological material obtained from a patient, and used in the assay, can be any of a variety of sample types. When derived from blood, the sample type can be, for example, derived from whole blood venipuncture, finger stick sample, and/or isolated serum. In some aspects, the sample type is obtained by swabbing one or more sores in the mouth, anal, and/or genital area. In still other aspects, the sample type is saliva, spinal fluid, urine, or tears, or a cheek swab.

According to various aspects, example assays can be designed to detect the IgG response, the IgM response or both. For instance, a sandwich type binding assay can include a first binding event where the blood sample is exposed to the G1-label and/or G2-label complex(es) under conditions such that antibodies present in the blood sample will bind to a region of the G1 and/or G2 sequence. The binding assay can also include a second binding event where the G1 and/or G2-label complex(es) bound to an antibody migrates (e.g., through capillary action) to a region including the G1 and/or G2 sequence(s) bound to a substrate. These two binding events lead to the formation of a new complex including two portions of the G1 and/or G2 sequence(s)—a first portion of the G1 and/or G2 sequence(s) that is bound to the substrate and a second portion of the G1 and/or G2 sequence(s) that is not bound and is linked to a label. Localized concentration of the complex in the area having the first portion of the G1 and/or G2 sequence(s) bound to the assay leads to production of a signal that can be identified visually as one possible mechanism for indicating the presence of antibodies against HSV-1 and/or HSV-2 in the sample.

In some embodiments, the assay is a multiplex-type assay that includes two regions: a first region having G1 sequence bound to the substrate and a second region having G2 sequence bound to the substrate, and, optionally, a control region. For example, in one aspect of this embodiment, there are two test lines, and one control line.

In one aspect of this embodiment, the G1-label complex can include a label having a certain emission profile that is different from the label included in the G2-label complex. While both of the complexes do not necessarily need to form (e.g., the sample may not include or have detectable levels of antibodies for both G1 and G2 sequence), the use of differently colored labels for each protein can allow for simultaneous and/or differential detection of viral infection using only a single assay.

In general, the antibody assay can be in the form of a test strip that includes a backing card, membrane (e.g., nitrocellulose), a conjugate pad, a sample pad, and a wick. The strip can be contained in a cassette or include other casings to protect the assay and/or the stability of material included in the assay.

In some implementations of the present disclosure, the assay may be in the form of a kit. The kit may include other reagents or materials for facilitating diagnosis, such as a chase buffer to facilitate movement of the sample along the strip, a result indicator to facilitate interpretation of the assay, a needle or stick to facilitate sample collection, and/or a sample collector such as a capillary pipet to transfer sample from the patient to the device.

An example implementation of the present disclosure can include a method for detecting the presence of HSV-1 and/or HSV-2 antibodies in a patient. The method can include applying a patient sample (e.g., blood) to a sample pad where capillary forces and the wick aid the movement of the sample through the sample pad and up the membrane. The sample fluid travels into a conjugate pad where the labeled conjugate proteins interact with the antibodies in the patient sample. Then, the fluid travels over the test line(s) on the membrane.

Generally, the HSV-1 and/or HSV-2 test lines includes a portion of the sequence for HSV-1 gG (e.g., G1 sequence) and/or HSV-2 gG (e.g., G2 sequence), respectively, that has already been striped and dried on the membrane. If positive, the patient antibodies that have bound to the G1-label complex will bind to the bound G1, and patient antibodies that have bound to the G2-label complex will bind to the bound G2, in a sandwich format. This will cause a visible change at the test line (e.g., formation of a colored or fluorescent area) that can be seen by the eye. If negative, no line will be visible. In some implementations, the sample fluid may continue to migrate up the membrane, which may further include a control line.

The control line can include antibodies or proteins already striped and dried on the membrane. The proteins or antibodies on the control line can be designed to bind to the labeled conjugate proteins, regardless of whether the labeled conjugate protein is bound to an antibody or not. For example, a labeled anti-chicken IgY striped and dried on the conjugate pad will be picked up with the sample fluid and travels along the membrane and over the control line. Chicken IgY has been striped and dried at the control line. The labeled anti-chicken IgY will bind to the chicken IgY at the control line.

Aspects of the example methods can include allowing the sample to run for a period of time, prior to making a determination of the result. For instance, wicking materials and/or peptide compositions may be adjusted to provide faster determining of antibody presence in the patient sample. In this manner, certain implementations may provide an advantage in antibody detection at point of care (e.g., within one hour) which may reduce costs and lead to less stress.

In another embodiment, treatment methods for HSV-1 or HSV-2 are disclosed. In this embodiment, a patient obtains a biological sample, tests the sample on the test strip, and, if the sample tests positive for HSV-1 and/or HSV-2 infection, the patient can be treated with antiviral agents and/or other types of treatment for HSV-1 and/or HSV-2 infection.

In still another embodiment, isolated peptides useful for carrying out the assays described herein are disclosed. The isolated peptides can include an amino acid sequence which comprises a portion of the G1 and/or G2 proteins.

In still another embodiment, isolated peptides useful for carrying out the assays described herein are disclosed. The isolated peptides can include an amino acid sequence which comprises a portion of the G1 (SEQ ID Nos. 1-4) and/or G2 (SEQ ID Nos. 5-7) proteins. For example, the isolated peptides can include greater than 90%, greater than 91%, greater than 92%, greater than 93%, greater than 94%, greater than 95%, greater than 96%, greater than 97%, greater than 98%, greater than 99%, or 100% of any of these sequences. In one embodiment, these isolated peptides can include 1, 2, 3, 4, or 5 conservative amino acid substitutions, so long as HSV-1 or HSV-2 antibodies, as appropriate, can bind to these peptide sequences in a manner sufficient for identifying the presence of the antibodies in a biological sample.

In some embodiments, the sequences comprise a His-tag.

In one embodiment, the isolated peptide is bound to a particle, such as a latex or a gold particle, and in some aspects of this embodiment, the isolated peptide is chemically or physically bound to the particle, for example, covalently bonded, ionically bonded, bonded by chemisorption or physisorption, bonded via functional linking groups or linker molecules, or bonded via hydrogen bonding, hydrophobic interaction, adsorption, absorption, or Van Der Waals attraction).

In another embodiment, the isolated peptide is chemically or physically bound to a substrate. In one embodiment, where the isolated peptide is bound to a substrate, the location where the isolated peptide is bound to the substrate is at the test line of an LFA. In another embodiment, the isolated peptide is also bound to the substrate at the control line of the LFA.

In another embodiment, the isolated peptide is bound to a particle, and also bound to a HSV-1 or HSV-2 antibody. In one aspect of this embodiment, the antibody in the “isolated peptide-particle-antibody conjugate” is also bound to an isolated peptide bound to a solid support. The conjugate (or complex) of the HSV-1 or HSV-2 antibody to a) the isolated peptide bound to a particle, and b) the isolated peptide bound to a solid support, is referred to as a “sandwich,” and is used in the sandwich assays described herein to indicate a user has tested positive for HSV-1 and/or HSV-2 infection.

### BRIEF DESCRIPTION OF THE DRAWINGS

Many aspects of the present disclosure will be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views. It should be recognized that these implementations and embodiments are merely illustrative of the principles of the present disclosure.

FIG. 1 illustrates an example configuration for running a lateral flow assay in accordance aspects of example implementations of the present disclosure.

FIG. 2 illustrates a table of example results for a lateral flow assay using different plasma samples in accordance with aspects of example implementations of the present disclosure.

FIG. 3A illustrates a cartoon of an example assay system. The system includes a portion of the gG protein for HSV-1 (G1) that is bound to a surface and another G1 sequence that is linked to a label (e.g., latex). The G1 and label can be linked using various polymers, peptides, or other systems. For example, as depicted, the linker can include two components (e.g., Biotin and Avidin).

FIG. 3B illustrates a cartoon of an example assay system. The system includes a portion of the gG protein for HSV-2 (G2) that is bound to a surface and another G2 sequence that is linked to a label (e.g., latex). The G2 and label can be linked using various polymers, peptides, or other systems. For example, as depicted, the linker can include two components (e.g., Biotin and Avidin).

FIG. 4A illustrates an example embodiment of an LFA when used with a blank sample depicting a sample area, a conjugate region, a test region, and a control region in accordance with example aspects of embodiments of the present disclosure.

FIG. 4B illustrates an example embodiment of an LFA when used with a positive sample depicting a sample area, a conjugate region, a test region, and a control region in accordance with example aspects of embodiments of the present disclosure.

FIG. 4C illustrates an example embodiment of an LFA used with a positive sample.

FIG. 5 illustrates a flow chart depicting the steps involved in detecting the presence or absence of an immune response in a biological sample using an example embodiment of an LFA in accordance with the present disclosure.

FIG. 6 illustrates an example embodiment of a kit including an LFA in accordance with example aspects of the present disclosure.

FIG. 7 illustrates an example embodiment of an LFA prior to application of the biological sample in accordance with example aspects of the present disclosure.

### DETAILED DESCRIPTION

Embodiments described herein can be understood more readily by reference to the following detailed description and examples. Systems, compositions and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

In general, the present disclosure is directed to lateral flow assays for detection of antibodies for HSV-1 and/or HSV-2. Aspects of such assays can include a solid support having at least two regions, each region defining an area of the solid support that includes an isolated peptide as described herein, and as shown in SEQ ID Nos 1-7.

In one embodiment, a device useful in lateral flow assays for detecting antibodies against HSV-1 and/or HSV-2 in a biological sample is disclosed. In some aspects of this embodiment, the biological sample is derived from blood, and can be obtained, for example, using venipuncture or finger stick. In other aspects, the biological sample can be, for example, a swab of an open sore, for example, in the mouth, anus, or genital areas, a urine sample, saliva sample, colostrum, breast milk, sample of cerebrospinal fluid, a tissue sample taken by swabbing a patient's cheek. The assay can be used, for example, by trained professionals in a clinical laboratory, hospital setting or a physician's office, or by individual patients at home, using a finger stick to obtain a blood sample, or obtaining other samples, for example, on a cotton swab.

The isolated peptides used in the assays (i.e., SEQ ID Nos. 1-4 for HSV-1 and 5-7 for HSV-2), and an analogs thereof) can be produced, for example, by expressing individual glycoprotein genes. The expression can be performed, for example, in baculovirus or CHO cells, and the peptides so produced can be isolated and purified.

In some embodiments, the LFA assay can also include a control line, and the primary function of the control line is to verify that the assay worked, i.e., that fluid, such as blood, plasma, or sera, flowed through the test strip and reached the control line. It is possible to use any colored particle bound to a compound that binds to any substance on the control line, such that the binding of the compound to the substance provides a colored signal on the control line, except that one cannot use particles bound to any compound that would also bind to the substance in the test line. In one embodiment, a colored particle is bound to anti-chicken IgY, and this anti-chicken IgY binds to chicken IgY present in the control line. Alternatively, a colored particle can be bound to chicken IgY, and this chicken IgY binds to anti-chicken IgY present in the control line. However, any control can be used where a first component (a label attached to a binding partner) is present in the assay before the control line, such as in the conjugation region, and migrates along the test strip with the other components in the assay, and the component binds to a second component, which second component is bound to the control line.

In some embodiments, the individual glycoproteins are available commercially, as are monoclonal antibodies and seropositive samples for each. The use of seropositive samples can serve as a reference control. That is, if a lab has a series of patient samples, and seropositive samples are mixed into the patient samples, if the lab fails to identify a known seropositive sample as a positive test, this will ideally alert the lab that the test is not being performed correctly.

In some embodiments, the assay is based on visual labels, conjugated to the isolated peptides, and interpreted without the use of a reader system. In other embodiments, the assay is quantitative, and is designed for use with a reader system. Multiple colored labels are available for evaluation, including colloidal gold, colored latex and cellulose nanobeads. In one non-limiting embodiment, the particles have a diameter of approximately 0.4 nm, and in one aspect of this embodiment, the particles are red latex particles. Other particle sizes can certainly be used, though the particles typically have a size less than 100 nm, and a size of at least approximately 0.2 nm. These particles, and their sizes, can be selected based on the requirements of the system for sensitivity and specificity.

In some embodiments, the assays are in a “sandwich” format using a colored signal reagent (in one aspect of these embodiments, red latex particles). The assay can detect, at a minimum, a patient's IgG response, but in some embodiments, can also detect a patients IgM response. This is important, as it may take a substantial amount of time for a patient to produce IgG, and the ability to detect IgM means that a patient may be able to be treated that much sooner, rather than waiting until IgG is present.

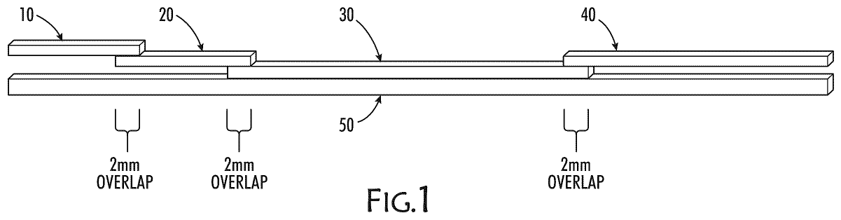

In one embodiment, as shown in FIG. 1, the assay strip includes a sample pad (10), a conjugate pad (20), a membrane (30) (e.g. a nitrocellulose membrane), and a wick pad (40), each of which is applied to a backing card (50). As shown in FIG. 1, one representative overlap between the various components is around 2 mm of overlap, though greater and lesser amounts of overlap can be used, or, in some embodiments, no overlap is necessary, so long as they do not interfere with the assay performance.

In some embodiments, the assay strips are contained in a standard high-performance plastic cassette, such as those available to DCN, and in other embodiments, a custom cassette is used, for example, to optimize performance.

In some embodiments, a chase or running buffer is used to facilitate the movement of the patient sample through the assay strip.

The overall system works by applying the patient sample to the sample pad, for example, in a well (not shown in FIG. 1, but typically present in a cassette into which the test strip is placed), whereby capillary forces and the wick pad aid the movement of the sample through the membrane. The sample fluid travels into the conjugate pad where labeled conjugate proteins interact with the antibodies in the patient sample, if present. In one embodiment, the labeled conjugate proteins comprise the isolated peptides of SEQ ID Nos. 1-7, or analogs thereof, bound, chemically or physically, to a particle, such as a colored latex or colloidal gold particle.

Then, the fluid travels over the test line on the membrane. The test line includes the HSV-1 and/or HSV-2 protein (i.e., the isolated peptides of SEQ ID Nos. 1-4 (HSV-1) or 5-7 (HSV-2), or analogs thereof) that has already been striped and dried on the membrane, and, optionally, attached to the membrane, for example, chemically or physically attached to the membrane.

If a patient's biological sample includes an antibody to HSV-1 and/or HSV-2, the antibody will bind to the labeled conjugate protein (i.e., the isolated peptide bound to the colored particle), and the antibody will further bind to the isolated peptide present on, and, optionally, bound to, the membrane at a location corresponding to the test line. A plurality of colored particles imparts a visible signal, such as a colored line, which indicates a positive test.

That is, if positive, the patient antibodies that have bound to the isolated peptide/particle conjugates will bind to the antibodies to HSV-1 and/or HSV-2 (in this case, the isolated peptide bound to the test strip at the test line) in a sandwich format, and the result is a visible red/brown line to be seen by the eye.

If the test is negative, no line will be visible, as no colored particles will bind (through binding of the colored particles to the isolated peptide, and the binding of the isolated peptide to the antibodies) to the test line.

The sample fluid will continue to run up the membrane, past the test line and over the control line. The control line includes a substance, which in one embodiment, is an antibody or protein (e.g. anti-chicken IgY), which is already striped and dried on the membrane. The isolated peptide/particle conjugates, not bound to a antibodies to HSV-1 and/or HSV-2, will have already been picked up by the sample fluid from the conjugate pad, and will attach to proteins or antibodies, such as anti-chicken IgY, on the control line. Regardless of whether the test is positive or negative, this line will show a visible line, such as a red/brown line.

The sample will run for a period of time, and, typically after the control line turns color, the result can be analyzed, in some embodiments, by a trained professional, and in other embodiments, by the patients themselves.

Embodiments described herein can be understood more readily by reference to the following detailed description and examples. Systems, compositions and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### I. Lateral Flow Assays

In general, the present disclosure is directed to lateral flow assays for detection of antibodies for antibodies for HSV-1 and/or HSV-2. Lateral flow tests (LFTs), also known as lateral flow immunochromatographic assays or rapid tests, are devices intended to detect the presence of a target substance, in this case, antibodies for HSV-1 and/or HSV-2, in a biological sample. These tests can be used in medical diagnostics for home testing, point of care testing, or laboratory use. These tests generally show results in around five to thirty minutes.

LFTs operate on the same principles of affinity chromatography as the enzyme-linked immunosorbent assays (ELISA). In essence, these tests run the liquid sample along the surface of a pad with reactive molecules that show a visual positive or negative result. The pads are based on a series of capillary beds, such as pieces of porous paper, microstructured polymer, or sintered polymer. Each of these pads has the capacity to transport fluid (e.g., urine, blood, plasma, serum, saliva and the like) spontaneously, though, in the assays described herein, blood, plasma and serum are the primary biological fluids that are evaluated.

The sample pad acts as a sponge and holds an excess of sample fluid. Once soaked, the fluid flows to the second conjugate pad in which the manufacturer has stored freeze dried bio-active particles called conjugates (see below) in a salt-sugar matrix. The conjugate pad contains all the reagents required for an optimized chemical reaction between the target molecule (e.g., an antigen) and its chemical partner (e.g., antibody) that has been immobilized on the particle's surface. This marks target particles as they pass through the pad and continue across to the test and control lines. The test line shows a signal, often a color as in pregnancy tests. The control line contains affinity ligands which show whether the sample has flowed through and the bio-molecules in the conjugate pad are active. After passing these reaction zones, the fluid enters the final porous material, the wick, that simply acts as a waste container.

LFTs can operate as either competitive or sandwich assays.

### Colored Particles

In principle, any colored particle can be used, however latex (blue color) or nanometer-sized particles of gold (red color) are most commonly used. The gold particles are red in color due to localized surface plasmon resonance. Fluorescent or magnetic labelled particles can also be used, however these require the use of an electronic reader to assess the test result.

### Sandwich Assays

Sandwich assays are generally used for larger analytes, because they tend to have multiple binding sites. As the sample migrates through the assay, it first encounters a conjugate, which is an antibody specific to the target analyte labelled with a visual tag, such as colloidal gold or colored latex particles. The antibodies bind to the target analyte within the sample and migrate together until they reach the test line.

The test line also contains immobilized antibodies specific to the target analyte, which bind to the migrated analyte bound conjugate molecules. The test line then presents a visual change due to the concentrated visual tag, hence confirming the presence of the target molecules. In some embodiments, the sandwich assays also have a control line, which will appear whether or not the target analyte is present to ensure proper function of the lateral flow pad.

### Competitive Assays

Competitive assays are generally used for smaller analytes since smaller analytes have fewer binding sites. The sample first encounters antibodies to the target analyte labelled with a visual tag (colored particles). The test line contains the target analyte fixed to the surface. When the target analyte is absent from the sample, unbound antibody will bind to these fixed analyte molecules, meaning that a visual marker will show. Conversely, when the target analyte is present in the sample, it binds to the antibodies to prevent them binding to the fixed analyte in the test line, and thus no visual marker shows. This differs from sandwich assays in that no band means the analyte is present.

### Quantitative Tests

Most LFTs are intended to operate on a purely qualitative basis. However, in some it is possible to measure the intensity of the test line to determine the quantity of analyte in the sample. Handheld diagnostic devices known as lateral flow readers are used by several companies to provide a fully quantitative assay result. By utilizing unique wavelengths of light for illumination in conjunction with either CMOS or CCD detection technology, a signal rich image can be produced of the actual test lines. Using image processing algorithms specifically designed for a particular test type and medium, line intensities can then be correlated with analyte concentrations. One such handheld lateral flow device platform is made by Detekt Biomedical L.L.C. Alternative non-optical techniques are also able to report quantitative assays results. One such example is a magnetic immunoassay (MIA) in the LFT form also allows for getting a quantified result. Reducing variations in the capillary pumping of the sample fluid is another approach to move from qualitative to quantitative results.

### Control Line

In some embodiments, the assays will incorporate a second line which contains a further antibody (one which is not specific to the analyte) that binds some of the remaining colored particles which did not bind to the test line. This confirms that fluid has passed successfully from the sample-application pad, past the test line. By giving confirmation that the sample has had a chance to interact with the test line, this increases confidence that a visibly-unchanged test line can be interpreted as a negative result (or that a changed test line can be interpreted as a negative result in a competitive assay). That is, if the control line of the test is blank, the test is invalid.

### Blood Plasma Extraction

Because the intense red color of hemoglobin interferes with the readout of colorimetric or optical detection-based diagnostic tests, blood plasma separation is a common first step to increase diagnostic test accuracy. Plasma can be extracted from whole blood via integrated filters or via agglutination. Accordingly, in some embodiments, the test strips include an integrated blood filter.

### Speed and Simplicity

In some embodiments, the tests can take as little as a few minutes to develop. Generally, there is a trade off between time and sensitivity: more sensitive tests may take longer to develop. The other key advantage of this format of test compared to other immunoassays is the simplicity of the test, by typically requiring little or no sample or reagent preparation.

### Wells

In some embodiments, the sample pad comprises a well. In some embodiments, the well has a sufficient volume to contain a solution containing the biological sample, which sample can optionally be diluted before being placed in the well.

In certain embodiments, the volume of the well ranges from about 1 μL to about 10 μL. In certain embodiments, the volume of the well ranges from about 1 μL to about 100 IL. In certain embodiments, the volume of the well ranges from about 1 μL to about 1000 μL. In certain embodiments, the volume of the well ranges from about 1 μL to about 5000 μL. In certain embodiments, the volume of the well ranges from about 1 mL to about 10 mL. In certain embodiments, the volume of the well ranges from about 1 mL to about 100 mL. In certain embodiments, the volume of the well ranges from about 1 mL to about 1000 mL.

In some embodiments, the well is located at a position of the LFA selected from a corner, an end, a center, a junction, an off-center, and a bend of the LFA. In some embodiments, the well comprises one or more pads selected from a salt pad, a probe pad, a polymer pad, and combinations thereof. In some embodiments, the well comprises a plurality of pads. In some embodiments, the first/second phase solutions separate and/or the target analyte concentrates as it flows through the plurality of pads. In some embodiments, the first/second phase solutions separate and/or the target analyte concentrates as it flows vertically through the plurality of pads. In some embodiments, the first/second phase solutions separate and/or the target analyte concentrates as it flows vertically through the plurality of pads due to gravity. In some embodiments, the first/second phase solutions separate and/or the target analyte concentrates as it flows vertically through the plurality of pads due to capillary action. In some embodiments, the well is a paper well. In some embodiments, the paper well is a three-dimensional paper structure holds a larger volume of sample compared to a typical paper strip used in LFA. In some embodiments, the paper well is composed of paper material that allows phase separation to occur and subsequent analyte concentration in the leading fluid. In some embodiments, the flow of the leading fluid is directed toward the absorbance pad that enables analyte detection.

In some embodiments, the device utilizes a “concentration-as-it-flows” mechanism, while further accelerating the flow and macroscopic phase separation utilizing gravitational force in the well. In some embodiments, the well provides a cross-sectional area sufficient to promote phase separation, since the first phase solution and the second phase solution may flow at a different speed due to differences in viscosity of the phase solutions, as well as differences in affinity for the paper material. In some embodiments, the well enhances or accelerates the phase separation and/or concentration of target analytes as the phase solution(s) travels through the well and emerges in the leading fluid. In some embodiments, the LFA test strip is connected directly to the well in a downstream position, so the concentrated analytes in the leading fluid first come in contact with the LFA strip and the detection step occurs concurrently with the concentration process, further reducing the overall assay time.

### LFA Design/Architecture

In some embodiments, the LFA strip has a width that does not vary from a first end to a second end. In some embodiments, the width is defined as a dimension perpendicular to the direction of flow within the LFA and in a plane of the length. In some embodiments, a first portion of the LFA strip has a first width and the second portion of the LFA strip has a second width, where the first width and the second width are different. In some embodiments, the first width is greater than the second width, while in other embodiments, the first width is less than the second width. In certain embodiments, it is contemplated that the LFA strip comprises more than two widths, e.g., the strip may continuously narrow, or may show progressive narrowing at three or more locations. In some embodiments, the first portion comprises the sample pad and the second portion comprises the detection zone. In some embodiments, a wider sample pad segment allows more target analyte in the sample to bind to the probe compared to an LFA strip wherein the width of the LFA strip does not vary. In some embodiments, a wider sample pad segment allows a greater volume of sample, and thus, more target analyte, to bind to the probe compared to an LFA strip wherein the width of the LFA strip does not vary.

In some embodiments, the LFA comprises a slope (e.g. a change in depth of the LFA along the length of the LFA). In some embodiments, wherein the LFA does not comprise a slope, a portion of the probe-analyte complex is left in the sample pad. In some embodiments, wherein the LFA comprises a slope, more probe analyte complex flows through the LFA than an LFA without a slope.

In some embodiments, the LFA is designed to be used with a probe that comprises or complexes with a magnetic/paramagnetic particle. In some embodiments, the LFA comprises a paper strip with a fork at the end of paper strip used to split the flow of the ATPS first and second phase solution. In some embodiments, the LFA detection zone is located on a prong of the fork, and magnets are located near or at the prong. The magnets concentrate the probe/probe-analyte complex into the fluid flowing into the LFA detection zone, which results in increased sensitivity of the diagnostic. Conversely, in some embodiments, the probe comprises the magnet or magnetic field, and the device comprises a magnetic particle or paramagnetic particle that is located near or at the prong.

In some embodiments, the LFA comprises a 3D architecture. In some embodiments, the LFA comprises layers of porous matrix resulting in a 3D architecture. In some embodiments, the 3D architecture integrates the ATPS with the LFA. In some embodiments, the ATPS has a long phase separation time, (e.g. a micellar ATPS) and phase separation time is improved by using a 3D architecture (e.g. increasing the height of the LFA strip). In some embodiments, the mixed phase solution separates into the first phase solution and the second phase solution as the phase solutions flow vertically through the LFA (e.g. through the layers of porous matrix).

In some embodiments, the LFA has a thickness (e.g. height, depth or vertical dimension). In some embodiments, the thickness is about 0.1 mm to about 30 cm. In some embodiments, the thickness is about 0.1 mm to about 1 mm, about 0.1 mm to about 10 mm, or about 0.1 mm to about 1 cm. In some embodiments, the thickness is about 1 mm to about 10 mm, about 1 mm to about 1 cm, about 1 mm to about 1.5 cm, about 1 mm to about 3 cm, about 1 mm to about 3.5 cm, about 1 mm to about 4 cm, about 1 mm to about 4.5 cm, about 1 mm to about 5 cm, about 1 mm to about 5.5 cm, about 1 mm to about 6 cm, about 1 mm to about 6.5 cm, about 1 mm to about 7 cm about 1 mm to about 7.5 cm, about 1 mm to about 8 cm, about 1 mm to about 8.5 cm, about 1 mm to about 9 cm, or about 1 mm to about 9.5 cm, or about 1 mm to about 10 cm. In some embodiments, the thickness is about 0.5 cm to about 5 cm.

### II. Isolated Peptides

In some embodiments, the assays described herein include a solid support having at least two regions, each region defining an area of the solid support that includes an isolated peptide. The isolated peptides are described in more detail below.

In some embodiments, the isolated peptide can include an amino acid sequence that comprises a portion of the HSV-1 gG protein and/or HSV-2 gG protein. Several non-limiting amino acid sequences in accordance with the present disclosure include:

G1-sequences:
(SEQ ID 1)
VPTNVSSTTQPQLQTTGRPSHEAPNMTQTGTTDSPTAISLTTPDHTPPMPSIGLEEE
EEEEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDT,
(SEQ ID 2)
MLLVNQSHQGFNKEHTSKMVSAIVLYVLLAAAAHSAFAVPTNVSSTTQPQLQTTGRPS
HEAPNMTQTGTTDSPTAISLTTPDHTPPMPSIGLEEEEEEEGAGDGEHLEGGDGTRDTLP
QSPGPAFPLAEDVEKDKPNRPVVPSPDPNNSPARPETSRPKTPPTIIGPLATRPTTRLTSKG
RPLVPTPQHTPLFSFLTASPALDTHHHHHH,
(SEQ ID 3)
MGWSCIILFLVATATGVHSVPTNVSSTTQPQLQTTGRPSHEAPNMTQTGTTDSPTAISLT
TPDHTPPMPSIGLEEEEEEEGAGDGEHLEGGDGTRDTLPQSPGPAFPLAEDVEKDKPNRP
VVPSPDPNNSPARPETSRPKTPPTIIGPLATRPTTRLTSKGRPLVPTPQHTPLFSFLTASPAL
DTHHHHHH,
(SEQ ID 4)
VPTNVSSTTQPQLQTTGRPSHEAPNMTQTGTTDSPTAISLTTPDHTPPMPSIGLEEE
EEEEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDT,
(SEQ ID 5)
VPTNVSSTTQPQLQTTGRPSHEAPNMTQTGTTDSPTAISLTTPDHTPPMPSIGLEEE
EEEEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDTHHHHHH,
(SEQ ID 6)
MGWSCIILFLVATATGVHSVPTNVSSTTQPQLQTTGRPSHEAPNMTQTGTTDSPT
AISLTTPDHTPPMPSIGLEEEEEEEGAGDGEHLEGGDGTRDTLPQSPGPAFPLAEDVEKD
KPNRPVVPSPDPNNSPARPETSRPKTPPTIIGPLATRPTTRLTSKGRPLVPTPQHTPLFSFLT
ASPALDT,
or
(SEQ ID 7)
MGWSCIILFLVATATGVHSVPTNVSSTTQPQLQTTGRPSHEAPNMTQTGTTDSPT
AISLTTPDHTPPMPSIGLEEEEEEEGAGDGEHLEGGDGTRDTLPQSPGPAFPLAEDVEKD
KPNRPVVPSPDPNNSPARPETSRPKTPPTIIGPLATRPTTRLTSKGRPLVPTPQHTPLFSFLT
ASPALDTHHHHHH.
G2 sequence(s)
(SEQ ID 8)
RALRTDPEGVDPDVRAPRTGRRLMALTEDTSSDSPTSAPEKTPLPVSATAMAPSV
DPSAEPTAPATTTPPDEMATQAATVAVTPEETAVASPPATASVESSPLPAAAAATPGAG
HTNTSSASAAKTPPTTPAPTTPPPTSTHATPRPTTPGPQTTPPGPATPGPVGASAAPTADSP
LTASPPATAPGPSAANVSVAATTATPGTRGTARTPPTDPKTHPHGPADAPPGSPAPPPPE
HRGGPEEFEGAGDGEPPEDDDSATGLAFRTPNPNKPPPARPGPIRPTLPPGILGPLAPNTP
RPPAQAPAKDMPSGPTPQHIPLFWFLTASPALD,
(SEQ ID 9)
MLLVNQSHQGFNKEHTSKMVSAIVLYVLLAAAAHSAFARALRTDPEGVDPDVR
APRTGRRLMALTEDTSSDSPTSAPEKTPLPVSATAMAPSVDPSAEPTAPATTTPPDEMAT
QAATVAVTPEETAVASPPATASVESSPLPAAAAATPGAGHTNTSSASAAKTPPTTPAPTT
PPPTSTHATPRPTTPGPQTTPPGPATPGPVGASAAPTADSPLTASPPATAPGPSAANVSVA
ATTATPGTRGTARTPPTDPKTHPHGPADAPPGSPAPPPPEHRGGPEEFEGAGDGEPPEDD
DSATGLAFRTPNPNKPPPARPGPIRPTLPPGILGPLAPNTPRPPAQAPAKDMPSGPTPQHIP
LFWFLTASPALDHHHHHH,
(SEQ ID 10)
MGWSCIILFLVATATGVHSRALRTDPEGVDPDVRAPRTGRRLMALTEDTSSDSPT
SAPEKTPLPVSATAMAPSVDPSAEPTAPATTTPPDEMATQAATVAVTPEETAVASPPATA
SVESSPLPAAAAATPGAGHTNTSSASAAKTPPTTPAPTTPPPTSTHATPRPTTPGPQTTPP
GPATPGPVGASAAPTADSPLTASPPATAPGPSAANVSVAATTATPGTRGTARTPPTDPKT
HPHGPADAPPGSPAPPPPEHRGGPEEFEGAGDGEPPEDDDSATGLAFRTPNPNKPPPARP
GPIRPTLPPGILGPLAPNTPRPPAQAPAKDMPSGPTPQHIPLFWFLTASPALDHHHHHH,
(SEQ ID 11)
MLLVNQSHQGFNKEHTSKMVSAIVLYVLLAAAAHSAFARALRTDPEGVDPDVR
APRTGRRLMALTEDTSSDSPTSAPEKTPLPVSATAMAPSVDPSAEPTAPATTTPPDEMAT
QAATVAVTPEETAVASPPATASVESSPLPAAAAATPGAGHTNTSSASAAKTPPTTPAPTT
PPPTSTHATPRPTTPGPQTTPPGPATPGPVGASAAPTADSPLTASPPATAPGPSAANVSVA
ATTATPGTRGTARTPPTDPKTHPHGPADAPPGSPAPPPPEHRGGPEEFEGAGDGEPPEDD
DSATGLAFRTPNPNKPPPARPGPIRPTLPPGILGPLAPNTPRPPAQAPAKDMPSGPTPQHIP
LFWFLTASPALDHHHHHH.
(SEQ ID 12)
MLLVNQSHQGFNKEHTSKMVSAIVLYVLLAAAAHSAFARALRTDPEGVDPDVR
APRTGRRLMALTEDTSSDSPTSAPEKTPLPVSATAMAPSVDPSAEPTAPATTTPPDEMAT
QAATVAVTPEETAVASPPATASVESSPLPAAAAATPGAGHTNTSSASAAKTPPTTPAPTT
PPPTSTHATPRPTTPGPQTTPPGPATPGPVGASAAPTADSPLTASPPATAPGPSAANVSVA
ATTATPGTRGTARTPPTDPKTHPHGPADAPPGSPAPPPPEHRGGPEEFEGAGDGEPPEDD
DSATGLAFRTPNPNKPPPARPGPIRPTLPPGILGPLAPNTPRPPAQAPAKDMPSGPTPQHIP
LFWFLTASPALD.
(SEQ ID 13)
RALRTDPEGVDPDVRAPRTGRRLMALTEDTSSDSPTSAPEKTPLPVSATAMAPSV
DPSAEPTAPATTTPPDEMATQAATVAVTPEETAVASPPATASVESSPLPAAAAATPGAG
HTNTSSASAAKTPPTTPAPTTPPPTSTHATPRPTTPGPQTTPPGPATPGPVGASAAPTADSP
LTASPPATAPGPSAANVSVAATTATPGTRGTARTPPTDPKTHPHGPADAPPGSPAPPPPE
HRGGPEEFEGAGDGEPPEDDDSATGLAFRTPNPNKPPPARPGPIRPTLPPGILGPLAPNTP
RPPAQAPAKDMPSGPTPQHIPLFWFLTASPALD.

In some embodiments, the isolated peptides used in the assays can consist substantially of the amino acid sequence set forth in any of SEQ ID Nos. 1-7 (for HSV-1) or 8-13 (for HSV-2). As used herein, “consisting substantially of” an amino acid sequence indicates that the isolated peptide includes the stated amino acid sequence with 5 or fewer amino acid additions or deletions to the N-terminus and/or the C-terminus. Thus, by way of example, for SEQ ID 4, which includes 56 amino acids, the isolated peptide can have an amino acid sequence that includes no less than 51 amino acids and no greater than 61 amino acids.

The isolated peptide can include an amino acid sequence (e.g., SEQ ID 4) that may further include one or more linker regions for binding the peptide to a region of the solid support. For instance, the one or more linker regions may include oligomers such as polyethylene glycol, acrylates, and/or peptides. Additionally or alternatively, the linker region may include a chemical modification such as thiolation, amidation, and/or esterification, which may be used to chemically attach the isolated peptide to the solid support.

The isolated peptide sequences provided herein (e.g., SEQ ID NOs: 1-7 for HSV-1 and 8-13 for HSV-2) may also have 1, 2, or 3 conservative mutations. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In another embodiment, the isolated peptide sequences are analogs of SEQ ID Nos. 1-13, which have at least 90% or greater sequence homology to any one or more of the polypeptide sequences of SEQ ID Nos. 1-7. More preferably, the peptide sequences have at least 95% or greater sequence homology, even more preferably at least 98% or greater sequence homology, and still more preferably at least 99% or greater sequence homology to any one or more of SEQ ID Nos. 1-7.

Methods for determining homology between nucleic acid and amino acid sequences are well known to those of ordinary skill in the art. For example, the “percent identity” of two amino acid sequences can be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Conjugates of the Isolated Peptide to Particles and/or Solid Supports

In one embodiment, the isolated peptide is bound to a particle, such as a latex or a gold particle, and in some aspects of this embodiment, the isolated peptide is chemically or physically bound to the particle, for example, covalently bonded, ionically bonded, bonded by chemisorption or physisorption, bonded via functional linking groups or linker molecules, or bonded via hydrogen bonding, hydrophobic interaction, adsorption, absorption, or Van Der Waals attraction.

In another embodiment, the isolated peptide is bound to a substrate, and in some aspects of this embodiment, the isolated peptide is chemically or physically bound to the substrate, for example, covalently bonded, ionically bonded, bonded by chemisorption or physisorption, bonded via functional linking groups or linker molecules, or bonded via hydrogen bonding, hydrophobic interaction, adsorption, absorption, or Van Der Waals attraction. In one embodiment, where the isolated peptide is bound to a substrate, the location where the isolated peptide is bound to the substrate is at the test line of an LFA.

In another embodiment, the isolated peptide is bound to a particle, and also bound to an HSV-1 or HSV-2 antibody. In one aspect of this embodiment, the antibody in the “isolated peptide-particle-antibody conjugate” is also bound to an isolated peptide bound to a solid support. The conjugate (or complex) of the HSV-1 or HSV-2 antibody to a) the isolated peptide bound to a particle, and b) the isolated peptide bound to a solid support, is referred to as a “sandwich,” and is used in the sandwich assays described herein to indicate a user has tested positive for HSV-1 or HSV-2 infection.

### Preparing Conjugates of GNPs and the Isolated Peptides

In some embodiments, rather than using latex particles, gold nanoparticles (GNPs) are used. Gold nanoparticles can be prepared to result in a clear, cherry-colored solution with particle sizes under 100 nm, such as around 25-30 nm in diameter. To prepare the GNPs, a solution of anti-Ge antibody can be incubated with a colloidal gold solution for a period of time, such as around 30 min, followed by the addition of thiolated-PEG5000, using a molar ratio of 3000:1 for PEG:GNP and an additional incubation of 30 min. To prevent nonspecific binding of other proteins to the surfaces of the colloidal gold, a bovine serum albumin (BSA) solution can be added to the mixture and mixed for an additional period of time, such as around 10 min. The resulting solution can be gently mixed during the incubation period. To remove free (unbound) antibodies, PEG, and BSA, the mixture can be subsequently centrifuged for an additional period of time, typically around 30 min, at a temperature of around 4° C. and 9,000 g.

The resulting pellet of GNPs can be washed, for example, with a 1% BSA solution. Finally, the recovered GNPs can be resuspended in a suitable buffer, such as a 0.1 M sodium borate buffer at a pH of around 9.

### Solid Supports

Solid supports used in LFAs are well known to those of skill in the art. In some embodiments, the solid supports are housed, for example, in a cassette, such as a plastic frame surrounding the test strip, with a well for the biological sample to be placed, and a window through which the two lines can be seen.

In some embodiments, the solid supports can include one of the at least two regions having the isolated peptide linked to the solid support. For instance, the region having the isolated peptide linked to the solid support can include the isolated peptide covalently or non-covalently bound to the solid support.

In some embodiments, the solid support includes one or more chemical functionalities which can be used to covalently link the solid support to one or more isolated peptides. For example, where the isolated peptides include a linker, the chemical functionalities on the solid support can be reacted with the linker region to bind the isolated peptide to the solid support.

Another aspect of some example solid supports can include one of the at least two regions having the isolated peptide not linked to the solid support. More particularly, the region having the isolated peptide not linked to the solid support can include the isolated peptide in contact with the solid support such that the isolated peptide can be solubilized when contacted with a liquid medium. For instance, the isolated peptide may be dried onto the solid support so that when the liquid medium is applied, some or all of the isolated peptide is solubilized in the liquid medium. Representative liquid media include various aqueous media such as water and salt/saline solutions. Additionally, some liquid media may include bodily fluids such as whole blood, blood serum, and/or lymph drainage.

For certain implementations, the isolated peptide not linked to the solid support can be fused to a heterologous protein, a detectable agent, a reactive agent, or combinations thereof. For example, the isolated peptide not linked to the solid support can include one or more functionalities to produce a detectable signal when incorporated as an immune assay. More particularly, in some implementations, the isolated peptide not linked to the solid support can include a heterologous protein that can act as or bind to a control marker. In this manner, as the isolated peptide not linked to the solid support is solubilized, it can bind to the control marker to indicate that the assay is working properly.

To produce a signal, the isolated peptide can also include a detectable agent and/or a reactive agent. The detectable agent can include an inert material such as a colored latex bead, colloidal metal (e.g., gold, silver, etc.), or other suitable products that upon binding to a region of the solid support, become concentrated such that visible signal is produced. The reactive agent can include a non-inert material that is chemically modified to produce a detectable signal such as fluorescence or a color change.

As an example for illustration, the isolated peptide not linked to the solid support can include a portion of the G1 protein sequence (e.g., SEQ ID 3) that is fused to Biotin, Avidin, and/or Streptavidin. The Biotin-Avidin system can further be linked to a detectible agent such as a colored latex bead, colloidal gold particle, and the like. In this manner, the isolated peptide not linked to the solid support includes a detectible agent linked to the amino acid sequence via a heterologous protein (e.g., Avidin or Streptavidin).

In some example implementations, the isolated peptide not linked to the solid support can have a concentration of no less than 10 ng/mm² and no greater than 600 ng/mm² based on the area of the region.

Additionally or alternatively, in certain example implementations, the isolated peptide linked to the solid support can have a concentration of no less than 1 ng/mm² and no greater than 500 ng/mm² based on the area of the region. For instance, the isolated peptide linked to the solid support can have a concentration of no less than 50 ng/mm² and no greater than 450 ng/mm², no less than 80 ng/mm² and no greater than 400 ng/mm², no less than 100 ng/mm² and no greater than 350 ng/mm², or no less than 120 ng/mm² and no greater than 350 ng/mm², based on the area of the region.

Solid Supports Comprising Isolated Peptides Rather than Bound Antibodies

In some aspects, the antibody detection assays include a solid support that is not directly linked to an antibody. While detecting antibodies is a primary use of certain embodiments, in other embodiments, it is not necessary to incorporate antibodies in the assays (e.g., via attachment to the solid support). In some aspects, the solid supports can include two regions which have an isolated peptide in accordance with the amino acid sequences disclosed herein. Further, the isolated peptide in each region may include the same amino acid sequence (e.g., both include one of SEQ ID Nos. 1-7 or 8-13) or the isolated peptide in each region may include different amino acid sequences (e.g., one includes one of SEQ ID Nos. 1-7 and the second includes one of SEQ ID Nos. 8-13, and all other combinations of these sequences).

### Solid Supports Comprising a Control Region

In other embodiments, the antibody detection assays include a solid support that further includes a control region. The control region can include a control agent that acts to bind to a portion of the isolated peptide not linked to the solid support.

In some aspects of these embodiments, the control region can include a protein that can dimerize or otherwise interact with the heterologous protein present on the isolated peptide not linked to the support.

In one representative example, the control agent can include Avidin, which may dimerize and/or bind to the Biotin-Avidin system to produce accumulation of the detectible agent in the control region.

As another example, the control agent can include a protein, such as an antibody (e.g., IgG, IgY, IgM, etc.) or a target of an antibody (e.g., anti-IgG, anti-IgY, anti-IgM, etc.) For instance, the isolated peptide not linked to the solid support can include the antibody (e.g., chicken IgY) bound directly to the isolated peptide as a fusion protein, or may include the antibody bound to the detectible agent, and the control region can include the target corresponding to the antibody (e.g., anti-chicken IgY).

### III. Representative Solid Supports

One aspect of some example solid supports can include one of the at least two regions having the isolated peptide linked to the solid support. For instance, the region having the isolated peptide linked to the solid support can include the isolated peptide covalently or non-covalently bound to the solid support. The isolated peptide can include an amino acid sequence (e.g., one of SEQ ID Nos. 1-13) that may further include one or more linker regions for binding the peptide to the region of the solid support. For instance, the one or more linker regions may include oligomers such as polyethylene glycol, acrylates, and/or peptides. Additionally or alternatively, the linker region may include a chemical modification such as thiolation, amidation, and/or esterification which may be used to covalently attach the isolated peptide to the solid support. For example, the solid support may also include one or more chemical functionalities which can be reacted with the linker region to bind (e.g., covalently attach) the isolated peptide to the solid support.

Another aspect of some example solid supports can include one of the at least two regions having the isolated peptide not linked to the solid support. More particularly, the region having the isolated peptide not linked to the solid support can include the isolated peptide in contact with the solid support such that the isolated peptide can be solubilized when contacted with a liquid medium. For instance, the isolated peptide may be dried onto the solid support so that when the liquid medium is applied, some or all of the isolated peptide is solubilized in the liquid medium. Example liquid media can include various aqueous media such as water and salt/saline solutions. Additionally, some liquid media may include bodily fluids such as whole blood, blood serum, finger stick, and/or lymph drainage.

For certain implementations, the isolated peptide not linked to the solid support can be fused to a heterologous protein, a detectable agent, a reactive agent, or combinations thereof. For example, the isolated peptide not linked to the solid support can include one or more functionalities to produce a detectable signal when incorporated as an immune assay. More particularly, in some implementations, the isolated peptide not linked to the solid support can include a heterologous protein that can act as or bind to a control marker. In this manner, as the isolated peptide not linked to the solid support is solubilized, it can bind to the control marker to indicate that the assay is working properly. To produce signal, the isolated peptide can also include a detectable agent and/or a reactive agent. The detectable agent can include an inert material such as a colored latex bead, colloidal metal (e.g., gold, silver, etc.), or other suitable products that upon binding to a region of the solid support, become concentrated such that visible signal is produced. The reactive agent can include a non-inert material that is chemically modified (e.g., via exposure to a pH modifier or to an excitation wavelength) to produce a detectable signal such as fluorescence or a color change.

As an example for illustration, the isolated peptide not linked to the solid support can include a portion of a G1 sequence (e.g., SEQ ID 3) that is fused to Biotin, Avidin, and/or Streptavidin. The Biotin-Avidin system can further be linked to a detectible agent such as a colored latex bead. In this manner, the isolated peptide not linked to the solid support includes a detectible agent linked to the amino acid sequence via a heterologous protein (e.g., Avidin or Streptavidin).

In some example implementations, the isolated peptide not linked to the solid support can have a concentration of no less than 10 ng/mm² and no greater than 600 ng/mm² based on the area of the region.

Additionally or alternatively, in certain example implementations, the isolated peptide linked to the solid support can have a concentration of no less than 1 ng/mm² and no greater than 500 ng/mm² based on the area of the region. For instance, the isolated peptide linked to the solid support can have a concentration of no less than 50 ng/mm² and no greater than 450 ng/mm², no less than 80 ng/mm² and no greater than 400 ng/mm², no less than 100 ng/mm² and no greater than 350 ng/mm², or no less than 120 ng/mm² and no greater than 350 ng/mm² based on the area of the region.

One aspect of some implementations can include antibody detection assays having a solid support that is not directly linked to an antibody. While detecting antibodies is a primary use of certain example implementations in accordance with the present disclosure, the incorporation of antibodies in the assays (e.g., via attachment to the solid support) is not required in some embodiments. Instead example solid supports can include two regions which have an isolated peptide in accordance with example amino acid sequences herein. Further, the isolated peptide in each region may include the same amino acid sequence (e.g., both include one of SEQ ID 1, 2, 3, 4, 5, 6, or 7 in the case of HSV-1, or 8, 9, 10, 11, 12, or 13 in the case of HSV-2) or the isolated peptide in each region may include different amino acid sequences (e.g., one includes SEQ ID 2 and the second includes SEQ ID 3).

Another aspect of some example implementations can include antibody detection assays having a solid support that further includes a control region. The control region can include a control agent that acts to bind to a portion of the isolated peptide not linked to the solid support.

For instance, the control region can include a protein that can dimerize or otherwise interact with the heterologous protein present on the isolated peptide not linked to the support. As one example for illustration, the control agent can include Avidin which may dimerize and/or bind to the Biotin-Avidin system to produce accumulation of the detectible agent in the control region. As another example, the control agent can include a protein such as an antibody (e.g., IgG, IgY, IgM, etc.) or a target of an antibody (e.g., anti-IgG, anti-IgY, anti-IgM, etc.) For instance, the isolated peptide not linked to the solid support can include the antibody (e.g., chicken IgY) bound directly to the isolated peptide as a fusion protein, or may include the antibody bound to the detectible agent, and the control region can include the target corresponding to the antibody (e.g., anti-chicken IgY).

Alternatively, the isolated peptide not linked to the solid support can include the target (e.g., anti-chicken IgY) bound directly to the isolated peptide as a fusion protein, or may include the target bound to the detectible agent, and the control region can include the antibody corresponding to the target (e.g., chicken IgY).

As should be understood, various combinations of the preceding disclosure may be produced and are within the scope of the present Application. For instance, one example implementation includes a solid support that includes: a wicking material for directing material flow along a direction of the solid support; a sample region comprising a first isolated peptide, wherein the first isolated peptide is not linked to the solid support, and wherein the first isolated peptide is fused to a heterologous protein; a detectable agent, a reactive agent, or combinations thereof; a test region positioned beyond the sample region along the direction of material flow, wherein the test region comprises a second isolated peptide linked to the solid support; and a control region positioned beyond the test region along the direction of material flow, wherein the control region comprises a control agent, and wherein each of the first isolated peptide and the second isolated peptide comprise an amino acid sequence set forth as SEQ ID 1.

For some implementations of the present disclosure, the first isolated peptide and the second isolated peptide can consist substantially of the amino acid sequence set forth as SEQ ID 2, 3, 4, 5, 6, or 7. As used herein, consisting substantially of an amino acid sequence indicates that the isolated peptide includes the stated amino acid sequence with 5 or fewer amino acid additions or deletions to the N-terminus and/or the C-terminus. Thus for SEQ ID 5, which includes 172 amino acids, the first isolated peptide and the second isolated peptide can have an amino acid sequence that includes no less than 162 amino acids and no greater than 182 amino acids. For SEQ ID 6, which includes 185 amino acids, the first isolated peptide and the second isolated peptide can have an amino acid sequence that includes no less than 175 amino acids and no greater than 195 amino acids. For SEQ ID 7, which includes 191 amino acids, the first isolated peptide and the second isolated peptide can have an amino acid sequence that includes no less than 181 amino acids and no greater than 201 amino acids.

Some additional, non-limiting, example embodiments are provided below.

In one embodiment, the solid support comprises at least two regions, each region defining an area of the solid support that includes an isolated peptide, wherein the isolated peptide comprises an amino acid sequence set forth as: SEQ ID 1, SEQ ID 2, SEQ ID 3, SEQ ID 4, SEQ ID 5, SEQ ID 6, SEQ ID 7, SEQ ID 8, SEQ ID 9, SEQ ID 10, SEQ ID 11, SEQ ID 12, SEQ ID 13, or a combination thereof.

In one aspect of this embodiment, the solid support is not directly linked to an antibody.

In another aspect of this embodiment, the solid support further comprises a control region, wherein the control region comprises a control agent.

In still another aspect of this embodiment, the solid support comprises at least three regions defining an area of the support including an isolated peptide, and wherein one of the at least three regions includes a the amino acid sequence as set forth in SEQ ID 1, 2, 3, 4, 5, 6, or 7, and another of the at least three regions includes the amino acid sequence as set forth in SEQ ID 8, 9, 10, 11, 12, or 13.

In one aspect of this embodiment, the isolated peptide consists substantially of the amino acid sequence set forth as: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, or SEQ ID No. 13.

In another aspect of this embodiment, for one of the at least two regions, the isolated peptide is linked to the solid support.

In yet another aspect of this embodiment, for one of the at least two regions, the isolated peptide is not linked to the solid support. For example, the isolated peptide not linked to the solid support can be fused to a heterologous protein; a detectable agent, such as a latex bead, a reactive agent, or combinations thereof.

In one non-limiting example, the isolated peptide not linked to the solid support can have a concentration of no less than 10 ng/mm² and no greater than 600 ng/mm² based on the area of the region. In another non-limiting example, the isolated peptide linked to the solid support has a concentration of no less than 1 ng/mm² and no greater than 500 ng/mm² based on the area of the region.

In another embodiment, the support comprises: a wicking material for directing material flow along a direction of the solid support; a sample region comprising a first isolated peptide, wherein the first isolated peptide is not linked to the solid support, and wherein the first isolated peptide is fused to a heterologous protein; a detectable agent, a reactive agent, or combinations thereof; a test region positioned beyond the sample region along the direction of material flow, wherein the test region comprises a second isolated peptide linked to the solid support; and a control region positioned beyond the test region along the direction of material flow, wherein the control region comprises a control agent, and wherein each of the first isolated peptide and the second isolated peptide comprise an amino acid sequence set forth as: (SEQ ID 1).

In one aspect of this embodiment, each of the first isolated peptide and the second isolated peptide consists substantially of the amino acid sequence set forth as: SEQ ID 2, SEQ ID 3, or SEQ ID 4.

In another embodiment, the support comprises: a wicking material for directing material flow along a direction of the solid support; a sample region comprising a first isolated peptide, wherein the first isolated peptide is not linked to the solid support, and wherein the first isolated peptide is fused to a heterologous protein; a detectable agent, a reactive agent, or combinations thereof; a test region positioned beyond the sample region along the direction of material flow, wherein the test region comprises a second isolated peptide linked to the solid support; and a control region positioned beyond the test region along the direction of material flow, wherein the control region comprises a control agent, and wherein each of the first isolated peptide and the second isolated peptide comprise an amino acid sequence set forth as: (SEQ ID 4).

In one aspect of this embodiment, each of the first isolated peptide and the second isolated peptide consists substantially of the amino acid sequence set forth as: SEQ ID 5, SEQ ID 6, or SEQ ID 7.

In another aspect of this embodiment, the detectable agent is a latex bead.

In another aspect of this embodiment, the first isolated peptide has a concentration of no less than 10 ng/mm² and no greater than 600 ng/mm² based on an area of the sample region.

### IV. Methods for Detecting Anti-HSV Antibodies

Methods for detecting anti-Herpes Simplex Virus (HSV 1 and/or 2) antibodies in a biological sample containing antibodies are also disclosed. In one embodiment, the method involves: contacting the biological sample with a solid support including an isolated peptide, wherein the isolated peptide includes an amino acid sequence set forth as: SEQ ID No. 1, SEQ ID No. 2, SEQ ID No. 3, SEQ ID No. 4, SEQ ID No. 5, SEQ ID No. 6, or SEQ ID No. 7 (for HSV-1) or SEQ ID No. 8, SEQ ID No. 9, SEQ ID No. 10, SEQ ID No. 11, SEQ ID No. 12 or SEQ ID No. 13 (for HSV-2) and where contacting is performed under conditions sufficient to form an immune complex between the isolated peptide and an antibody present in the biological sample.

The presence or absence of the immune complex can then be detected, preferably visually, though other suitable methods may be used. Detection of the immune complex provides indication of the presence of anti-HSV antibodies in the biological sample.

One aspect of example methods can include detecting the presence or absence of the immune complex based on the presence of a color change on the solid support (e.g., the formation of a red, yellow or other colored shape, such as a line).

Another aspect of example methods can include incubating the assay after contacting the biological sample with the solid support for a time period. For implementations of the present disclosure, the incubation time is generally less than about 1 hour such as no less than 0.5 minutes and no greater than 60 minutes, no less than 1 minutes and no greater than 55 minutes, no less than 5 minutes and no greater than 50 minutes, no less than 10 minutes and no greater than 50 minutes, no less than 15 minutes and no greater than 45 minutes, or no less than 20 minutes and no greater than 40 minutes.

With reference now to the Drawings, the present disclosure contemplates antibody detection assays that can be formed on a solid support. The solid support can include two or more regions which include an isolated peptide. For instance, an example solid support can include a backing card on which the assay is formed. The assay can include a sample region for applying a biological sample, optionally, a membrane region for reducing unnecessary components in the biological sample (e.g., cells), a binding region which includes the isolated peptide not bound to the solid support for creating an immune complex with antibody present in the biological sample, and a test region which includes the isolated peptide bound to the solid support. As the soluble immune complex migrates to the test region, a sandwich capture causes the immune complex to concentrate at the test region to form a detectable signal. FIG. 1 illustrates one example design for a solid support. The solid support includes a sample region (10 mm MF1), a binding region (10 mm 6614), a membrane region (25 mm CN95), and a test region (21 mm 222). The overall layout can be organized so that fluid applied to the sample region migrates laterally to the binding region prior to reaching the test region. Fluid migration allows soluble complexes such as the immune complex to reach the test region and provide a detectable signal. As should be understood, numbers illustrated in FIG. 1 are for example purposes only and need not constrain the size of each region.

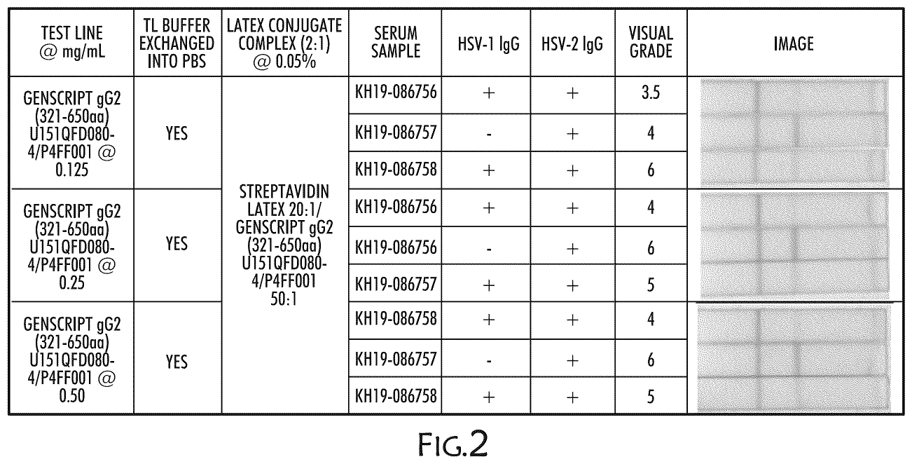

Referring now to FIG. 2, an example test strip system is depicted for an HSV-2 IgG LFA comparing test lines of gG2 antigen expressed by Genscript (lot U151QFD080-4/P4FF001) striped @ 0.125, 0.25, and 0.5 mg/mL with negative serum samples (n=1).

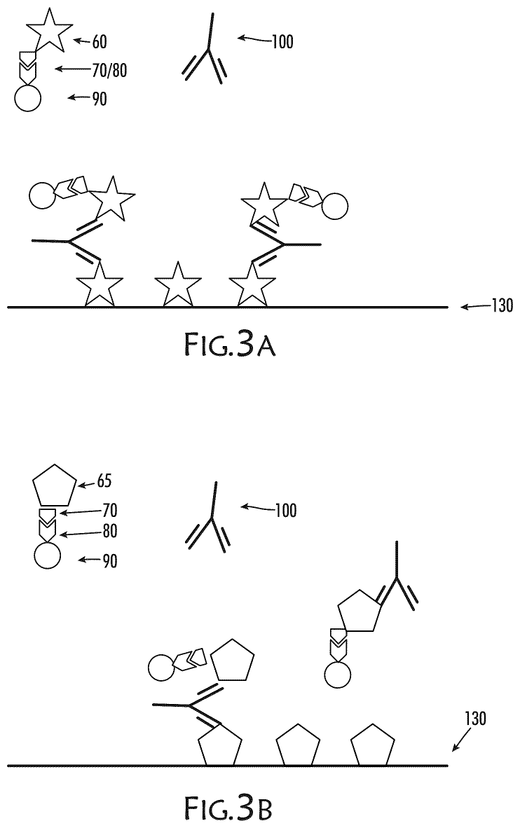

FIG. 3A illustrates a cartoon of an example sandwich assay system at the region of the test line (160). The system includes a portion of the G1 protein (60, depicted as a blue star) that is adsorbed to a test strip surface (e.g. nitrocellulose membrane) and another isolated G1 protein (60) that is linked to a label (e.g., a latex particle) (90, depicted as a red circle), creating a G1-latex complex. The G1 and label can be linked using various polymers, peptides, chemical reactions, or other systems. For example, as depicted in FIG. 3A, the linker can include two components (e.g., Biotin and Avidin) (70 and 80, depicted as red and green connected symbols).

The Conjugate Pad (150) is striped with the G1 (60)-latex (90) complex, in addition to the Chicken IgY (120)-Latex (90) complex. The assay works when the sample that contains anti-G1 IgG and/or IgM (collectively referred to as (100) binds with the G1, also referred to herein as the isolated peptide of SEQ ID Nos. 1-4, or analog thereof (60)-latex (90) complex, forming an anti-G1-G1-latex complex. The sample solution is then wicked up the test strip to the region of the test line (160), where the anti-G1-G1-latex complex binds to the isolated gE peptide (60) adsorbed to the surface (130). The test line region will turn a visible color (e.g. red) after enough of the complexes are bound.

Instead of, or in addition to the G1 protein, a G2 protein, such as those in SEQ ID Nos. 5-7 can be used. If both are used, then the G1 (60)-latex (90) complex is present in one test line, and the G2 (60)-latex (90) complex is present in a second test line. FIG. 3B is an example where a G2 protein (65) is used.

Referring now to FIG. 3B, this figure depicts an example assay testing system that includes an unbound isolated peptide. The unbound isolated peptide can include a conjugate of an amino acid sequence of the gG protein for HSV-2 (G2) having a linker (e.g., Biotin-Avidin) bound to a detectible agent (e.g., Latex bead). The G2-label complex can be bound by Anti-G2 antibodies present in a biological sample to form an immune complex. The immune complex can then migrate to an assay region having isolated peptide bound to a solid surface. The isolated peptide bound to the solid surface can include the amino acid sequence of the gG protein for HSV-2 such as SEQ ID 8, 9, 10, 11, 12, or 13.

FIGS. 3A and 3B depict example assay testing systems that include an unbound isolated peptide. The unbound isolated peptide can include a conjugate of an amino acid sequence of the gG protein for HSV-1 (G1) having a linker (e.g., Biotin-Avidin) bound to a detectible agent (e.g., Latex bead). The G1-label complex can be bound by Anti-G1 antibodies present in a biological sample to form an immune complex. The immune complex can then migrate to an assay region having isolated peptide bound to a solid surface. The isolated peptide bound to the solid surface can include the amino acid sequence of the gG protein for HSV-1 such as SEQ ID 1, 2, 3, 4, 5, 6, or 7.

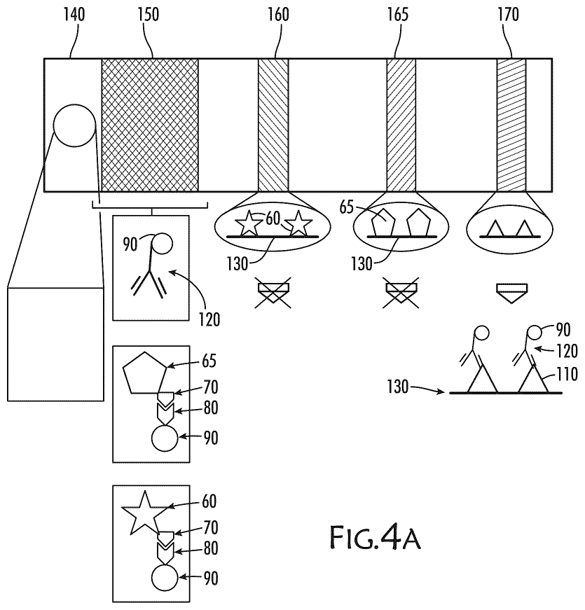

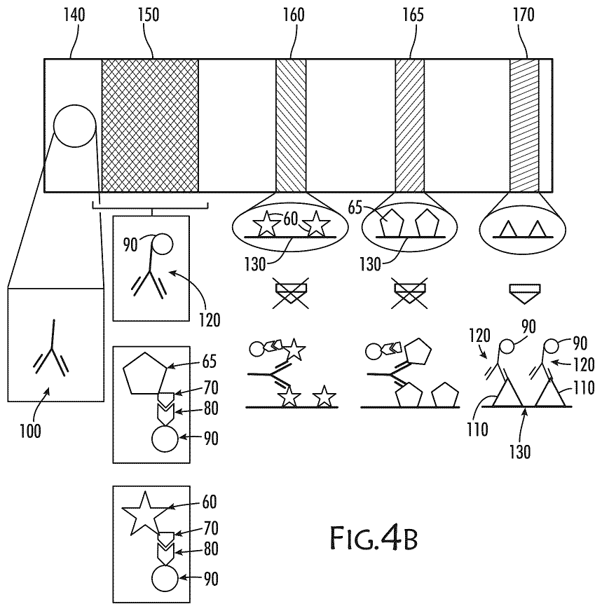

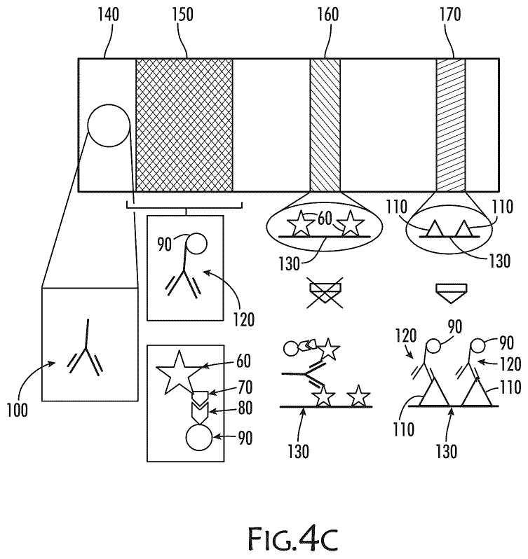

FIG. 4A illustrates an example embodiment of a lateral flow assay formed on a solid support in accordance with the present disclosure. The figure depicts application of a blank sample (unfilled circle, 140) onto a sample area, or well, of the LFA. The sample migrates through the conjugate region, which includes a peptide not linked to the solid support that is fused to a heterologous protein; a detectable agent, a reactive agent, or combinations thereof. In this case, the peptide (star) is fused to a detectible agent (circle) via one or more heterologous proteins (chevrons(s)).

Additionally, in some implementations and as depicted, the assay can also include a second peptide (pentagon) that is fused to a detectible agent (circle) via one or more heterologous proteins. For implementations that include the two different proteins, one can include a G1 sequence (star) and the other a G2 sequence (pentagon), or vice-versa, allowing for the differential detection of antibodies for HSV-1 and HSV-2.

The fluid migration solubilizes some of the peptide not linked to the solid support so that it moves up to a test region which includes the peptide linked to the solid support (star in contact with dark line) and further moves up to the control region, which includes a marker bound to the solid support (triangle in contact with dark line). The marker in the control region can be specific for binding one or more of the heterologous protein(s). In this manner, the blank sample can lead to a signal forming at the control line, but not forming at the test line.

In the blank sample in FIG. 4A, anti-chicken IgG (120) is bound (via biotin/streptavidin) to a particle/label (90). The chicken IgY (120) and particle/label (90) can be linked using various polymers, peptides, chemical reactions, or other systems. There are two test lines, (160 for G1 and 165 for G2) which include the G1 (60)-latex (90) complex (60) and the G2 (65)-latex (90) complex, respectively. The anti-chicken IgY (110, black triangle) is striped on the control line (170). The Conjugate Pad (150) is striped with the Chicken IgY (120)-Latex particle (90) complex, in addition to the G1 (60)-latex particle (90) complex and G2 (65)-latex particle (90) complex.

Once the sample solution is wicked through the region of the control line (170), the Chicken IgY (120)-Latex particle (90) complex will bind to the anti-chicken IgY (110) on the surface (130). Since there are no antibodies for HSV-1 or HSV-2 in this example, only the test line turns a visible color.

In FIG. 4B, the Conjugate Pad (150) is also striped with the Chicken IgY (120)-Latex particle (90) complex, in addition to the G1 (60)-latex particle (90) complex and G2 (65)-latex particle (90) complex. In this example, the sample includes both HSV-1 and HSV-2 antibodies (both depicted as 100). Accordingly, after the test is run, the test line regions (160 and 165) and the control line region (170) will turn a visible color (e.g. red) after enough of the complexes are bound.

FIG. 4C illustrates another example embodiment of a lateral flow assay. FIG. 4C more particularly illustrates a non-multiplex assay that only includes one peptide-label complex (e.g., G1-label complex (i.e., 60-90)). The biological sample can contain antibodies (100) specific to a region of the G1. For example, the biological sample can include antibodies (Ab) for HSV-1. As the biological sample migrates through the conjugate region (150), an antibody peptide conjugate is formed which migrates further up to the test region (160). At the test region, the peptide linked to the solid support binds the second arm of the antibody resulting in the antibody peptide complex concentrating at the test line (160) to form a positive signal. The test line (160) includes a G1 sequence (60) bound to the support and would indicate the presence of antibodies for HSV-1 in the biological sample. As depicted, a control line (170) can also be included to ensure that the sample is migrating far enough to lead to a positive signal at the test line. In another embodiment, the peptide-label complex is a G2-label complex (i.e., 65-90).

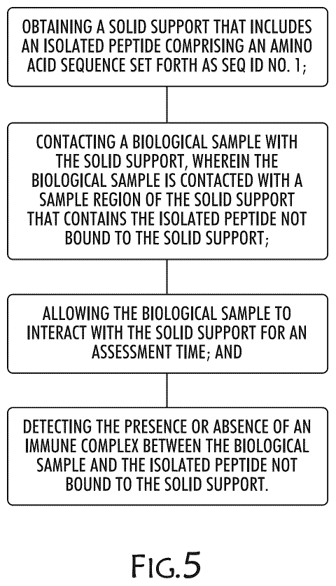

FIG. 5 illustrate a flow chart depicting an example method for using a LFA to detect the presence of an antibody in a biological sample. The method can include a sequence of steps that can be performed in a specified order or that may be rearranged. Further, the depicted method is provided for example only, different variations that include other steps or omit certain steps are also within the scope of the present disclosure. In the depicted embodiment, the method can include obtaining a solid support that includes an isolated peptide comprising an amino acid sequence set forth as SEQ ID. 1. The method can also include contacting a biological sample (e.g., blood and/or plasma) with the solid support, wherein the biological sample is contacted with a sample region of the solid support that contains (or is adjacent to a region containing) the isolated peptide not bound to the solid support. The method can also include allowing the biological sample to interact with the solid support for an assessment time. In general, aspects of example LFAs in accordance with the present disclosure provide for reduced assessment time compared to other assessment techniques such as ELISA. As such, for certain implementations, the assessment time can be no less than 1 minute and no greater than 60 minutes. Finally, the method can include detecting the presence or absence of an immune complex between the biological sample and the isolated peptide not bound to the solid support. The immune complex, if present, may be formed between antibody present in the biological sample and the isolated peptide having the amino acid sequence set forth as SEQ ID 1.

In another embodiment, a method for detecting anti-Herpes Simplex Virus antibodies in a biological sample containing antibodies is disclosed, where the method involves: contacting the biological sample with a solid support including an isolated peptide, wherein the isolated peptide comprises an amino acid sequence set forth as: SEQ ID 1, and wherein contacting is performed under conditions sufficient to form an immune complex between the isolated peptide and an antibody present in the biological sample, and detecting the presence or absence of the immune complex, wherein the presence of the immune complex indicates anti-HSV antibodies are present in the biological sample.

In one aspect of this embodiment, the isolated peptide consists substantially of the amino acid sequence set forth as:
SEQ ID 2, or SEQ ID 3.

In another embodiment, the immune complex, if present, may be formed between antibody present in the biological sample and the isolated peptide having the amino acid sequence set forth as SEQ ID 4.

In another embodiment, a method for detecting anti-Herpes Simplex Virus antibodies in a biological sample containing antibodies is disclosed, where the method involves: contacting the biological sample with a solid support including an isolated peptide, wherein the isolated peptide comprises an amino acid sequence set forth as: SEQ ID 1, and wherein contacting is performed under conditions sufficient to form an immune complex between the isolated peptide and an antibody present in the biological sample, and detecting the presence or absence of the immune complex, wherein the presence of the immune complex indicates anti-HSV antibodies are present in the biological sample.

In one aspect of this embodiment, the isolated peptide consists substantially of the amino acid sequence set forth as:
SEQ ID 5, SEQ ID 6, or SEQ ID 7.

In another aspect of this embodiment, a first portion of the isolated peptide is linked to the solid support, and/or a second portion of the isolated peptide is not linked to the solid support. By way of example, the isolated peptide not linked to the solid support can be fused to a heterologous protein; a detectable agent, a reactive agent, or combinations thereof. The detectable agent can be a latex bead, and detecting the presence of or absence of the immune complex can be determined based on presence of a color change on the solid support.

### V. Kits

In some embodiments, the assay may be in the form of a kit. The kit may include other reagents or materials for facilitating diagnosis such as a chase buffer to facilitate movement of the sample along the strip, a result indicator to facilitate interpretation of the assay, and/or a needle or stick to facilitate sample collection, where the biological sample is blood, or a swab or other suitable sample collector where the biological sample is other than blood. The kit may also include pertinent information for obtaining a blood sample, performing the assay using the blood or other biological sample, and interpreting the results of the assay. In addition to these elements, the kit can include a capillary tube to draw blood from the sample in a measured amount for adding to the well of the LFA. The kit can also include a chase buffer to add to the blood sample in the well. A chase buffer is typically a salt-based buffer, such as physiological saline and phosphate buffered saline.

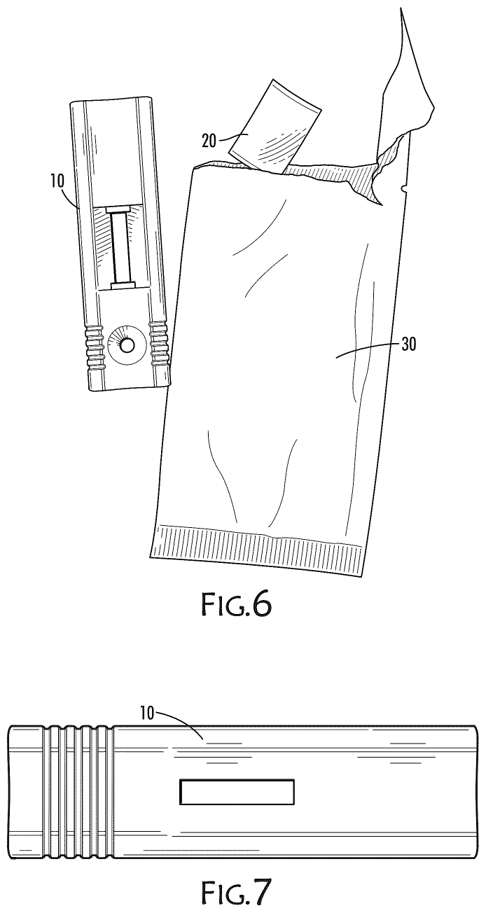

FIG. 6 illustrates an example embodiment of a kit including an LFA (10) in accordance with example aspects of the present disclosure, along with a drying agent (20) and packaging (30), and FIG. 7 illustrates an example embodiment of an LFA (10) prior to application of a biological sample.

### VI. Theranostic Applications

In some embodiments, once a patient has been diagnosed as having a Varicella Zoster infection, the patient is then treated with an antiviral medication. Although other agents can be used, several agents commonly used against HSV-1 or HSV-2, and which can be used in the theranostic methods described herein, are discussed below.

When a patient is first diagnosed with herpes (whether HSV-1 or HSV-2), using the assays described herein, the patient may be prescribed, for example, a 7- to 10-day course of antiviral therapy. If symptoms do not improve in that time, the patient may continue with the antiviral course for a longer duration. Following the initial treatment, a patient may be treated with intermittent or suppressive treatments. One representative suppressive therapy is a once-daily administration of Valacyclovir (Valtrex), which significantly reduces transmission among couples of HSV-2, or genital herpes. Other commonly used drugs include Acyclovir (Zovirax), Famciclovir (Famvir), Docosanol (Abreva), Denavir and (Penciclovir).

The present invention will be better understood with reference to the following non-limiting examples with reference to the foregoing drawings.

### Examples

The present examples provide aspects of embodiments of the present disclosure. These examples are not meant to limit embodiments solely to such examples herein, but rather to illustrate some possible implementations.

### Method:

### Making Strips:

1. Laminate membrane on 60 mm backing card 16 mm from the bottom.
2. Laminate 222 wick pad flush to the top of the backing card and with a 2 mm overlap with the nitrocellulose membrane.
3. Cut the bottom, unlaminated section of the backing card off.
4. Cut into 4 mm strips.

### Lateral Flow Assay—Batch Format:

1. Prepare 4 mL run buffers:
   a) 1×PBS, 1% Tergitol, 1% Casein
      i. 10×PBS—0.4 mL
      ii. 25% Tergitol—0.16 mL
      iii. 6% casein—0.667 mL
      iv. Water—2.773 mL
   b) 1×PBS, 1% Tween20, 1% Casein
      i. 10×PBS—0.4 mL
      ii. 10% Tween20—0.4 mL
      iii. 6% casein—0.667 mL
      iv. Water—2.533 mL
2. Prepare 1 mL 0.5% casein in 50 mM borate:
   a) 6% casein—166.7 μL
   b) 50 mM borate—833 μL
3. Sonicate CNB conjugates.
4. Dilute all of the conjugated as described in the below table:

TABLE ##

| | CNB | CNB | CNB | 0.5% | |
|---|---|---|---|---|---|
| CNB gG | Stock % | Final % | Stock μL | Casein μL | Total μL |
| Arvys A | 0.43 | 0.03 | 5.23 | 69.8 | 75 |
| Arvys B | 0.43 | 0.03 | 5.23 | 69.8 | 75 |
| Genscript U016 | 0.43 | 0.03 | 5.23 | 69.8 | 75 |
| Genscript U489 | 0.46 | 0.03 | 4.89 | 70.1 | 75 |

5. Add 5 μL of serum sample to glass tubes.
6. Add 31 μL of run buffer to each tube.
   a) Note: all assays ran with 1% tergitol run buffer unless otherwise noted.
7. Add 4 μL of diluted conjugate to each tube.
8. Vortex to mix.
9. Add half strips with striped gG membrane to each tube.
10. Allow assay to run for 10 minutes.
11. Record the results.

### Lateral Flow Assay—Sequential Format:

1. To glass tubes, add 27 μL of run buffer.
   a) Note: all assays ran with 1% tergitol run buffer unless otherwise noted.
2. To the tubes, add 3 μL of diluted conjugate.
3. Vortex to mix.
4. Dilute serum samples 4-fold in run buffer.
   a) Ex: per assay 5 μL of serum+15 μL of run buffer
   b) Note: all assays ran with 1% tergitol run buffer unless otherwise noted.
5. Add 20 μL of diluted serum to new, clean glass tubes.
6. Add half strips with striped gG membrane to each tube with diluted serum sample.
7. Allow sample to run for 3 minutes.
8. Transfer strips to the tubes containing conjugate.
9. Allow assay to run for 10 more minutes (13 minutes total).
10. Record the results.

### 2. HSV Conjugate

Streptavidin is conjugated to 0.4 nm Red Latex particles at a mass ratio of 20:1 or 40:1 (Beads:Protein)
HSV gG (1-350aa) from Genscript, Zeba column exchanged into 1×PBS was biotinylated with NHS-PEG12-Biotin at a molar ratio of 25:1 (Biotin:Protein)
HSV gG-biotin was complexed with Streptavidin-Latex at a 2:1 molar ratio (HSV gG-biotin:Streptavidin-Latex)
Chicken IgY is conjugated to 0.4 nm Red Latex particles at a mass ratio of 40:1 (Beads:Protein)
Streptavidin-Latex/HsV gG-biotin conjugate complex was prepared in 50 mM Tris, 1% casein (7 day cured), 0.5% Tergitol, 10% sucrose, and 2% Trehalose to final concentration of 0.075% solids with Chicken IgY-Latex at a final concentration of 0.01% solids.

### 3. HSV Conjugate Pad

Ahlstrom 6614 glass fiber pad is cut to 10 mm.

Conjugate solution with 0.075% Streptavidin-Latex/HSV gG-biotin conjugate complex was sprayed on to the 6614 polyester conjugate pad with 1 pass of 10 μL/cm towards the bottom of the pad.

### 4. HSV Sample Pad

GE MF1 glass fiber pad is cut to 10 mm.

### 5. Wick Pad

Ahlstrom 222 is cut to 21 mm and placed onto the backing card at the top with a 2 mm overlap with the membrane.

6. Final strips are assembled with a 21 mm Ahlstrom 222 wicking pad placed flush with top edge of the 60 mm backing card and overlapping the top edge of the CN95 membrane by 2 mm. The 10 mm 6614 conjugate pad is placed overlapping the bottom edge of the CN95 membrane by 2 mm. An 10 mm MF1 Sample pad is placed overlapping the bottom edge of the 10 mm conjugate pad by 2 mm. Strips are cut to 4.9 mm wide, and placed in to the custom MICA 200 cassette.

7. Chase buffer is 12 mM Sodium Phosphate, 0.6M NaCl, and 1% Tergitol.

### 8. Test Procedure

Pipette 10 μL of whole blood sample into the sample port.

Allow samples to fully absorb into the sample pad for 30 seconds.

Pipette 70 μL of chase buffer to the sample port.

Allow strips to run for 10 minutes before reading or interpreting results.

### 9. Layout

TABLE 1
HSV IgG Lateral Flow Assay Parameters

| | | | Location from | | | |
|---|---|---|---|---|---|---|
| LFA Strip | Material | Length | bottom of Strip | | Thickness | |
| Membrane | Sartorius CN95 | 25 mm | 16 | mm | 240-270 | μm |
| Conjugate Pad | Ahlstrom 6614 | 10 mm | 8 | mm | 420 | μm |
| Sample Pad | GE MF1 | 10 mm | 0 | mm | 367 | μm |
| Wick Pad | Ahlstrom 222 | 21 mm | 39 | mm | 830 | μm |

| | | | | | |
|---|---|---|---|---|---|
| Backing Card | Lohmann | 60 mm | N/A | 254 | μm |

### 10. Test Principal

The lateral flow test works as a sandwich assay and is housed in a cassette. The test cassette consists substantially of a test strip that contains a sample pad, conjugate pad, membrane, and wick pad. The purpose of each component is as follows: 1) the patient sample is applied to the sample pad that contains a blood cell separator to facilitate flow of the sample fluid up the test strip; 2) a conjugate pad containing HSV gG recombinant antigen that is attached to biotin and is complexed with Streptavidin conjugated to Red Colored Latex particles forming a HSV gG-biotin: Streptavidin-Latex complex. Chicken IgY protein conjugated to Red Latex particles is also incorporated with the gG-biotin:Streptavidin-Latex complex solution and is sprayed on towards the bottom of the conjugate pad and placed in to a cassette; 3) a nitrocellulose membrane strip containing a striped donkey anti-Chicken IgG line (control line) and a striped HSV gG recombinant antigen line (test line); and 4) a wick pad that draws off liquid by capillary action.

When a correct volume of test blood, sera or plasma specimen is dispensed into the sample pad of the test strip, the specimen migrates by capillary action along the test strip. The anti-HSV (type 1 and/or type 2) IgG, if present in the specimen, will bind to the HSV gG-biotin:Streptavidin-Latex complex and further migrate to bind to the HSV gG antigen (test line) forming a red-colored test line, indicating a HSV IgG positive test result, or no line will be visible to indicate a negative test result. The sample fluid will continue to run up the membrane over the control line. The chicken IgY-Latex complex will bind to anti-Chicken IgG protein on the control line and cause a visible red line to become visible regardless of whether the sample is positive or negative for HSV IgG. The results can be read within 10-20 minutes after the sample was added to the sample pad.

All references referred to herein are hereby incorporated by reference for all purposes.

Many modifications and other embodiments of the invention will come to the mind of one skilled in the art having the benefit of the teachings presented in the foregoing descriptions and the associated drawings. Therefore, it is to be understood that the inventive concepts described herein are not to be limited to the specific embodiments disclosed, and that the modifications and embodiments are intended to be included within the scope of the dependent claims.

While certain exemplary embodiments of the inventive concept have been described and shown in the accompanying drawings, it is to be understood that such embodiments are merely illustrative of and not restrictive, and that the embodiments of the inventive concept not be limited to the specific constructions and arrangements shown and described, since various other modifications may occur to those ordinarily skilled in the art.

1. A solid support comprising at least two regions, each region defining an area of the solid support that includes an isolated peptide, wherein the isolated peptide comprises an amino acid sequence set forth as:
(SEQ ID No. 4)
VPTNVSSTTQPQLQTTGRPSHEAPNMTQTGTTDSPTAISLTTPDHTPPMPSIGLEEEEE
EEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDT,
(SEQ ID No. 5)
VPTNVSSTTQPQLQTTGRPSHEAPNMTQTGTTDSPTAISLTTPDHTPPMPSIGLEEEEE
EEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDTHHHHHH,
(SEQ ID No. 6)
MGWSCIILFLVATATGVHSVPTNVSSTTQPQLQTTGRPSHEAPNMTQTGTTDSPTAIS
LTTPDHTPPMPSIGLEEEEEEEGAGDGEHLEGGDGTRDTLPQSPGPAFPLAEDVEKDK
PNRPVVPSPDPNNSPARPETSRPKTPPTIIGPLATRPTTRLTSKGRPLVPTPQHTPLFSFL
TASPALDT,
(SEQ ID No. 7)
MGWSCIILFLVATATGVHSVPTNVSSTTQPQLQTTGRPSHEAPNMTQTGTTDSPTAIS
LTTPDHTPPMPSIGLEEEEEEEGAGDGEHLEGGDGTRDTLPQSPGPAFPLAEDVEKDK
PNRPVVPSPDPNNSPARPETSRPKTPPTIIGPLATRPTTRLTSKGRPLVPTPQHTPLFSFL
TASPALDTHHHHHH,
(SEQ ID No. 11)
MLLVNQSHQGFNKEHTSKMVSAIVLYVLLAAAAHSAFARALRTDPEGVDPDVRAPR
TGRRLMALTEDTSSDSPTSAPEKTPLPVSATAMAPSVDPSAEPTAPATTTPPDEMATQ
AATVAVTPEETAVASPPATASVESSPLPAAAAATPGAGHTNTSSASAAKTPPTTPAPT
TPPPTSTHATPRPTTPGPQTTPPGPATPGPVGASAAPTADSPLTASPPATAPGPSAANV
SVAATTATPGTRGTARTPPTDPKTHPHGPADAPPGSPAPPPPEHRGGPEEFEGAGDGE
PPEDDDSATGLAFRTPNPNKPPPARPGPIRPTLPPGILGPLAPNTPRPPAQAPAKDMPS
GPTPQHIPLFWFLTASPALDHHHHHH,
(SEQ ID No. 12)
MLLVNQSHQGFNKEHTSKMVSAIVLYVLLAAAAHSAFARALRTDPEGVDPDVRAPR
TGRRLMALTEDTSSDSPTSAPEKTPLPVSATAMAPSVDPSAEPTAPATTTPPDEMATQ
AATVAVTPEETAVASPPATASVESSPLPAAAAATPGAGHTNTSSASAAKTPPTTPAPT
TPPPTSTHATPRPTTPGPQTTPPGPATPGPVGASAAPTADSPLTASPPATAPGPSAANV
SVAATTATPGTRGTARTPPTDPKTHPHGPADAPPGSPAPPPPEHRGGPEEFEGAGDGE
PPEDDDSATGLAFRTPNPNKPPPARPGPIRPTLPPGILGPLAPNTPRPPAQAPAKDMPS
GPTPQHIPLFWFLTASPALD,
(SEQ ID No. 13)
RALRTDPEGVDPDVRAPRTGRRLMALTEDTSSDSPTSAPEKTPLPVSATAMAPSVDP
SAEPTAPATTTPPDEMATQAATVAVTPEETAVASPPATASVESSPLPAAAAATPGAG
HTNTSSASAAKTPPTTPAPTTPPPTSTHATPRPTTPGPQTTPPGPATPGPVGASAAPTA
DSPLTASPPATAPGPSAANVSVAATTATPGTRGTARTPPTDPKTHPHGPADAPPGSPA
PPPPEHRGGPEEFEGAGDGEPPEDDDSATGLAFRTPNPNKPPPARPGPIRPTLPPGILGP
LAPNTPRPPAQAPAKDMPSGPTPQHIPLFWFLTASPALD,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 4-7 and 11-13, and
d) functional groups for covalently attaching the isolated peptide sequences to a solid support,
and combinations thereof,
optionally wherein the solid support is not directly linked to an antibody,
optionally wherein the solid support further comprises a control region, and wherein the control region comprises a control agent, and
optionally wherein the isolated peptide consists essentially of the amino acid sequence set forth as:
(SEQ ID No. 4)
VPTNVSSTTQPQLQTTGRPSHEAPNMTQTGTTDSPTAISLTTPDHTPPMPSIGLEEEEE
EEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDT,
(SEQ ID No. 5)
VPTNVSSTTQPQLQTTGRPSHEAPNMTQTGTTDSPTAISLTTPDHTPPMPSIGLEEEEE
EEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDTHHHHHH,
(SEQ ID No. 6)
MGWSCIILFLVATATGVHSVPTNVSSTTQPQLQTTGRPSHEAPNMTQTGTTDSPTAIS
LTTPDHTPPMPSIGLEEEEEEEGAGDGEHLEGGDGTRDTLPQSPGPAFPLAEDVEKDK
PNRPVVPSPDPNNSPARPETSRPKTPPTIIGPLATRPTTRLTSKGRPLVPTPQHTPLFSFL
TASPALDT,
(SEQ ID No. 7)
MGWSCIILFLVATATGVHSVPTNVSSTTQPQLQTTGRPSHEAPNMTQTGTTDSPTAIS
LTTPDHTPPMPSIGLEEEEEEEGAGDGEHLEGGDGTRDTLPQSPGPAFPLAEDVEKDK
PNRPVVPSPDPNNSPARPETSRPKTPPTIIGPLATRPTTRLTSKGRPLVPTPQHTPLFSFL
TASPALDTHHHHHH,
(SEQ ID No. 11)
MLLVNQSHQGFNKEHTSKMVSAIVLYVLLAAAAHSAFARALRTDPEGVDPDVRAPR
TGRRLMALTEDTSSDSPTSAPEKTPLPVSATAMAPSVDPSAEPTAPATTTPPDEMATQ
AATVAVTPEETAVASPPATASVESSPLPAAAAATPGAGHTNTSSASAAKTPPTTPAPT
TPPPTSTHATPRPTTPGPQTTPPGPATPGPVGASAAPTADSPLTASPPATAPGPSAANV
SVAATTATPGTRGTARTPPTDPKTHPHGPADAPPGSPAPPPPEHRGGPEEFEGAGDGE
PPEDDDSATGLAFRTPNPNKPPPARPGPIRPTLPPGILGPLAPNTPRPPAQAPAKDMPS
GPTPQHIPLFWFLTASPALDHHHHHH,
(SEQ ID No. 12)
MLLVNQSHQGENKEHTSKMVSAIVLYVLLAAAAHSAFARALRTDPEGVDPDVRAPR
TGRRLMALTEDTSSDSPTSAPEKTPLPVSATAMAPSVDPSAEPTAPATTTPPDEMATQ
AATVAVTPEETAVASPPATASVESSPLPAAAAATPGAGHTNTSSASAAKTPPTTPAPT
TPPPTSTHATPRPTTPGPQTTPPGPATPGPVGASAAPTADSPLTASPPATAPGPSAANV
SVAATTATPGTRGTARTPPTDPKTHPHGPADAPPGSPAPPPPEHRGGPEEFEGAGDGE
PPEDDDSATGLAFRTPNPNKPPPARPGPIRPTLPPGILGPLAPNTPRPPAQAPAKDMPS
GPTPQHIPLFWFLTASPALD,
or
(SEQ ID No. 13)
RALRTDPEGVDPDVRAPRTGRRLMALTEDTSSDSPTSAPEKTPLPVSATAMAPSVDP
SAEPTAPATTTPPDEMATQAATVAVTPEETAVASPPATASVESSPLPAAAAATPGAG
HTNTSSASAAKTPPTTPAPTTPPPTSTHATPRPTTPGPQTTPPGPATPGPVGASAAPTA
DSPLTASPPATAPGPSAANVSVAATTATPGTRGTARTPPTDPKTHPHGPADAPPGSPA
PPPPEHRGGPEEFEGAGDGEPPEDDDSATGLAFRTPNPNKPPPARPGPIRPTLPPGILGP
LAPNTPRPPAQAPAKDMPSGPTPQHIPLFWFLTASPALD,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 4-7 and 11-13, and
d) functional groups for covalently attaching the isolated peptide sequences to a solid support.

2. (canceled)

3. The solid support of claim 1, wherein for one of the at least two regions, the isolated peptide is linked to the solid support, optionally wherein the isolated peptide linked to the solid support has a concentration of no less than 1 ng/mm² and no greater than 500 ng/mm² based on the area of the region.

4. The solid support of claim 1, wherein for one of the at least two regions, the isolated peptide is not linked to the solid support, optionally wherein the isolated peptide not linked to the solid support is fused to a heterologous protein: a detectable agent, a reactive agent, or combinations thereof, optionally wherein the detectable agent is a latex bead or a colloidal gold particle, and optionally wherein the isolated peptide not linked to the solid support has a concentration of no less than 10 ng/mm² and no greater than 600 ng/mm² based on the area of the region.

5-10. (canceled)

11. The solid support of claim 1, wherein the solid support comprises at least three regions defining an area of the support including an isolated peptide, and wherein one of the at least three regions include the amino acid sequence as set forth in any one of SEQ ID Nos. 4, 5, 6, or 7, and another of the at least three regions includes the amino acid sequence as set forth in any one of SEQ ID Nos. 11, 12 or 13.

12. A solid support, wherein the support comprises:
a wicking material for directing material flow along a direction of the solid support;
a sample region comprising a first isolated peptide, wherein the first isolated peptide is not linked to the solid support, and wherein the first isolated peptide is fused to a heterologous protein; a detectable agent, a reactive agent, or combinations thereof,
a test region positioned beyond the sample region along the direction of material flow, wherein the test region comprises a second isolated peptide linked to the solid support; and
a control region positioned beyond the test region along the direction of material flow, wherein the control region comprises a control agent, and wherein
each of the first isolated peptide and the second isolated peptide comprise an amino acid sequence set forth as:
(SEQ ID No. 4)
VPTNVSSTTQPQLQTTGRPSHEAPNMTQTGTTDSPTAISLTTPDHTPPM
PSIGLEEEEEEEGAGDGEHLEGGDGTRDTLPQSPGPAFPLAEDVEKDKP
NRPVVPSPDPNNSPARPETSRPKTPPTIIGPLATRPTTRLTSKGRPLVP
TPQHTPLFSFLTASPALDT,
and analogues thereof wherein the first and second isolated peptide sequences comprise one or more of:
a) up to 5 amino acids at the C and/or at the N terminal ends,
b) one, two or three conservative amino acid substitutions,
c) 98% or greater sequence homology to SEQ ID No. 4, and
d) functional groups for covalently attaching the first and/or second isolated peptide sequences to a solid support,
optionally wherein the detectable agent is a latex bead or a gold particle,
optionally wherein the first isolated peptide has a concentration of no less than 10 ng/mm2 and no greater than 600 ng/mm2 based on an area of the sample region, and
optionally wherein each of the first isolated peptide and the second isolated peptide consists substantially of the amino acid sequence set forth as:
(SEQ ID No. 5)
VPTNVSSTTQPQLQTTGRPSHEAPNMTQTGTTDSPTAISLTTPDHTPPMPSIGLEEEEE
EEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDTHHHHHH,
(SEQ ID No. 6)
MGWSCIILFLVATATGVHSVPTNVSSTTQPQLQTTGRPSHEAPNMTQTGTTDSPTAIS
LTTPDHTPPMPSIGLEEEEEEEGAGDGEHLEGGDGTRDTLPQSPGPAFPLAEDVEKDK
PNRPVVPSPDPNNSPARPETSRPKTPPTIIGPLATRPTTRLTSKGRPLVPTPQHTPLFSFL
TASPALDT,
or
(SEQ ID No. 7)
MGWSCIILFLVATATGVHSVPTNVSSTTQPQLQTTGRPSHEAPNMTQTGTTDSPTAIS
LTTPDHTPPMPSIGLEEEEEEEGAGDGEHLEGGDGTRDTLPQSPGPAFPLAEDVEKDK
PNRPVVPSPDPNNSPARPETSRPKTPPTIIGPLATRPTTRLTSKGRPLVPTPQHTPLFSFL
TASPALDTHHHHHH,
and analogues thereof wherein the first and second isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 4-7, and
d) functional groups for covalently attaching the first and/or second isolated peptide sequences to a solid support.

13-15. (canceled)

16. A method for detecting anti-Herpes Simplex Virus antibodies in a biological sample containing antibodies, comprising:
contacting the biological sample with a solid support including an isolated peptide of claim 1 under conditions sufficient to form an immune complex between the isolated peptide and an antibody present in the biological sample, and detecting the presence or absence of the immune complex, wherein the presence of the immune complex indicates anti-HSV antibodies are present in the biological sample.

17-21. (canceled)

22. The method of claim 16, further comprising administering a therapeutic agent to a patient that tests positive for HSV-1 and/or HSV-2 infection, wherein the therapeutic agent is effective at treating the symptoms of a HSV-1 and/or HSV-2 infection, or in treating the underlying viral infection, optionally wherein the therapeutic agent is selected from the group consisting of Acyclovir Zovirax), Famciclovir (Famvir), Docosanol (Abreva), Denavir and (Penciclovir).

23. (canceled)

24. An isolated peptide having an amino acid sequence set forth as:
(SEQ ID No. 4)
VPTNVSSTTQPQLQTTGRPSHEAPNMTQTGTTDSPTAISLTTPDHTPPMPSIGLEEEEE
EEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDT,
(SEQ ID No. 5)
VPTNVSSTTQPQLQTTGRPSHEAPNMTQTGTTDSPTAISLTTPDHTPPMPSIGLEEEEE
EEGAGDGEHLEGGDGTRDTLPQSPGPAFPLAEDVEKDKPNRPVVPSPDPNNSPARPE
TSRPKTPPTIIGPLATRPTTRLTSKGRPLVPTPQHTPLFSFLTASPALDTHHHHHH,
(SEQ ID No. 6)
MGWSCIILFLVATATGVHSVPTNVSSTTQPQLQTTGRPSHEAPNMTQTGTTDSPTAIS
LTTPDHTPPMPSIGLEEEEEEEGAGDGEHLEGGDGTRDTLPQSPGPAFPLAEDVEKDK
PNRPVVPSPDPNNSPARPETSRPKTPPTIIGPLATRPTTRLTSKGRPLVPTPQHTPLFSFL
TASPALDT,
(SEQ ID No. 7)
MGWSCIILFLVATATGVHSVPTNVSSTTQPQLQTTGRPSHEAPNMTQTGTTDSPTAIS
LTTPDHTPPMPSIGLEEEEEEEGAGDGEHLEGGDGTRDTLPQSPGPAFPLAEDVEKDK
PNRPVVPSPDPNNSPARPETSRPKTPPTIIGPLATRPTTRLTSKGRPLVPTPQHTPLFSFL
TASPALDTHHHHHH.
(SEQ ID No. 11)
MLLVNQSHQGFNKEHTSKMVSAIVLYVLLAAAAHSAFARALRTDPEGVDPDVRAPR
TGRRLMALTEDTSSDSPTSAPEKTPLPVSATAMAPSVDPSAEPTAPATTTPPDEMATQ
AATVAVTPEETAVASPPATASVESSPLPAAAAATPGAGHTNTSSASAAKTPPTTPAPT
TPPPTSTHATPRPTTPGPQTTPPGPATPGPVGASAAPTADSPLTASPPATAPGPSAANV
SVAATTATPGTRGTARTPPTDPKTHPHGPADAPPGSPAPPPPEHRGGPEEFEGAGDGE
PPEDDDSATGLAFRTPNPNKPPPARPGPIRPTLPPGILGPLAPNTPRPPAQAPAKDMPS
GPTPQHIPLFWFLTASPALDHHHHHH,
(SEQ ID No. 12)
MLLVNQSHQGFNKEHTSKMVSAIVLYVLLAAAAHSAFARALRTDPEGVDPDVRAPR
TGRRLMALTEDTSSDSPTSAPEKTPLPVSATAMAPSVDPSAEPTAPATTTPPDEMATQ
AATVAVTPEETAVASPPATASVESSPLPAAAAATPGAGHTNTSSASAAKTPPTTPAPT
TPPPTSTHATPRPTTPGPQTTPPGPATPGPVGASAAPTADSPLTASPPATAPGPSAANV
SVAATTATPGTRGTARTPPTDPKTHPHGPADAPPGSPAPPPPEHRGGPEEFEGAGDGE
PPEDDDSATGLAFRTPNPNKPPPARPGPIRPTLPPGILGPLAPNTPRPPAQAPAKDMPS
GPTPQHIPLFWFLTASPALD,
or
(SEQ ID No. 13)
RALRTDPEGVDPDVRAPRTGRRLMALTEDTSSDSPTSAPEKTPLPVSATAMAPSVDP
SAEPTAPATTTPPDEMATQAATVAVTPEETAVASPPATASVESSPLPAAAAATPGAG
HTNTSSASAAKTPPTTPAPTTPPPTSTHATPRPTTPGPQTTPPGPATPGPVGASAAPTA
DSPLTASPPATAPGPSAANVSVAATTATPGTRGTARTPPTDPKTHPHGPADAPPGSPA
PPPPEHRGGPEEFEGAGDGEPPEDDDSATGLAFRTPNPNKPPPARPGPIRPTLPPGILGP
LAPNTPRPPAQAPAKDMPSGPTPQHIPLFWFLTASPALD,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 4-7 and 11-13,
d) one or more functional groups for covalently attaching the isolated peptide sequences to a solid support, and
e) a solid support to which the isolated peptide is attached, through the functional group or
f) a particle to which the isolated peptide is attached, either covalently or non-covalently,
optionally, further comprising an HSV-1 or HSV-2 antibody bound to the isolated peptide bound to the particle, and, optionally, wherein the HSV-1 or HSV-2 antibody is bound to a) an isolated peptide bound to a particle, and b) an isolated peptide bound to a solid support.

25-26. (canceled)

27. A solid support comprising at least two regions, each region defining an area of the solid support that includes an isolated peptide, wherein the isolated peptide comprises an amino acid sequence set forth as:
(SEQ ID No. 1)
VPTNVSSTTQPQLQTTGRPSHEAPNMTQTGTTDSPTAISLTTPDHTPPMPSIGL
EEEEEEEGAGDGEHLEGGDGTRDTLPQSPGPAFPLAEDVEKDKPNRPVVPSPDPNNS
PARPETSRPKTPPTIIGPLATRPTTRLTSKGRPLVPTPQHTPLFSFLTASPALDT,
(SEQ ID No. 2)
MLLVNQSHQGFNKEHTSKMVSAIVLYVLLAAAAHSAFAVPTNVSSTTQPQL
QTTGRPSHEAPNMTQTGTTDSPTAISLTTPDHTPPMPSIGLEEEEEEEGAGDGEHLEG
GDGTRDTLPQSPGPAFPLAEDVEKDKPNRPVVPSPDPNNSPARPETSRPKTPPTIIGPL
ATRPTTRLTSKGRPLVPTPQHTPLFSFLTASPALDTHHHHHH,
(SEQ ID No. 3)
MGWSCIILFLVATATGVHSVPTNVSSTTQPQLQTTGRPSHEAPNMTQTGTTDS
PTAISLTTPDHTPPMPSIGLEEEEEEEGAGDGEHLEGGDGTRDTLPQSPGPAFPLAEDV
EKDKPNRPVVPSPDPNNSPARPETSRPKTPPTIIGPLATRPTTRLTSKGRPLVPTPQHTP
LFSFLTASPALDTHHHHHH,
(SEQ ID No. 8)
RALRTDPEGVDPDVRAPRTGRRLMALTEDTSSDSPTSAPEKTPLPVSATAMAP
SVDPSAEPTAPATTTPPDEMATQAATVAVTPEETAVASPPATASVESSPLPAAAAATP
GAGHTNTSSASAAKTPPTTPAPTTPPPTSTHATPRPTTPGPQTTPPGPATPGPVGASAA
PTADSPLTASPPATAPGPSAANVSVAATTATPGTRGTARTPPTDPKTHPHGPADAPPG
SPAPPPPEHRGGPEEFEGAGDGEPPEDDDSATGLAFRTPNPNKPPPARPGPIRPTLPPGI
LGPLAPNTPRPPAQAPAKDMPSGPTPQHIPLFWFLTASPALD,
(SEQ ID No. 9)
MLLVNQSHQGFNKEHTSKMVSAIVLYVLLAAAAHSAFARALRTDPEGVDPD
VRAPRTGRRLMALTEDTSSDSPTSAPEKTPLPVSATAMAPSVDPSAEPTAPATTTPPD
EMATQAATVAVTPEETAVASPPATASVESSPLPAAAAATPGAGHTNTSSASAAKTPP
TTPAPTTPPPTSTHATPRPTTPGPQTTPPGPATPGPVGASAAPTADSPLTASPPATAPGP
SAANVSVAATTATPGTRGTARTPPTDPKTHPHGPADAPPGSPAPPPPEHRGGPEEFEG
AGDGEPPEDDDSATGLAFRTPNPNKPPPARPGPIRPTLPPGILGPLAPNTPRPPAQAPA
KDMPSGPTPQHIPLFWFLTASPALDHHHHHH,
(SEQ ID No. 10)
MGWSCIILFLVATATGVHSRALRTDPEGVDPDVRAPRTGRRLMALTEDTSSD
SPTSAPEKTPLPVSATAMAPSVDPSAEPTAPATTTPPDEMATQAATVAVTPEETAVAS
PPATASVESSPLPAAAAATPGAGHTNTSSASAAKTPPTTPAPTTPPPTSTHATPRPTTP
GPQTTPPGPATPGPVGASAAPTADSPLTASPPATAPGPSAANVSVAATTATPGTRGTA
RTPPTDPKTHPHGPADAPPGSPAPPPPEHRGGPEEFEGAGDGEPPEDDDSATGLAFRT
PNPNKPPPARPGPIRPTLPPGILGPLAPNTPRPPAQAPAKDMPSGPTPQHIPLFWFLTAS
PALDHHHHHH,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 1-3 and 8-10, and
d) functional groups for covalently attaching the isolated peptide sequences to a solid support,
and combinations thereof,
optionally, wherein for one of the at least two regions, the isolated peptide is linked to the solid support, or wherein for one of the at least two regions, the isolated peptide is not linked to the solid support, and, optionally, where the isolated peptide is not linked to the solid support, it is fused to a heterologous protein: a detectable agent, a reactive agent, or combinations thereof, optionally wherein the detectable agent is a latex bead or a colloidal gold particle,
optionally wherein the isolated peptide is not linked to the solid support, it has a concentration of no less than 10 ng/mm² and no greater than 600 ng/mm² based on the area of the region, or wherein the isolated peptide is linked to the solid support, it has a concentration of no less than 1 ng/mm² and no greater than 500 ng/mm² based on the area of the region, and
optionally wherein the isolated peptide consists essentially of the amino acid sequence set forth as:
(SEQ ID No. 1)
VPTNVSSTTQPQLQTTGRPSHEAPNMTQTGTTDSPTAISLTTPDHTPPMPSIGL
EEEEEEEGAGDGEHLEGGDGTRDTLPQSPGPAFPLAEDVEKDKPNRPVVPSPDPNNS
PARPETSRPKTPPTIIGPLATRPTTRLTSKGRPLVPTPQHTPLFSFLTASPALDT,
(SEQ ID No. 2)
MLLVNQSHQGFNKEHTSKMVSAIVLYVLLAAAAHSAFAVPTNVSSTTQPQL
QTTGRPSHEAPNMTQTGTTDSPTAISLTTPDHTPPMPSIGLEEEEEEEGAGDGEHLEG
GDGTRDTLPQSPGPAFPLAEDVEKDKPNRPVVPSPDPNNSPARPETSRPKTPPTIIGPL
ATRPTTRLTSKGRPLVPTPQHTPLFSFLTASPALDTHHHHHH,
(SEQ ID No. 3)
MGWSCIILFLVATATGVHSVPTNVSSTTQPQLQTTGRPSHEAPNMTQTGTTDS
PTAISLTTPDHTPPMPSIGLEEEEEEEGAGDGEHLEGGDGTRDTLPQSPGPAFPLAEDV
EKDKPNRPVVPSPDPNNSPARPETSRPKTPPTIIGPLATRPTTRLTSKGRPLVPTPQHTP
LFSFLTASPALDTHHHHHH,
(SEQ ID No. 8)
RALRTDPEGVDPDVRAPRTGRRLMALTEDTSSDSPTSAPEKTPLPVSATAMAP
SVDPSAEPTAPATTTPPDEMATQAATVAVTPEETAVASPPATASVESSPLPAAAAATP
GAGHINTSSASAAKTPPTTPAPTTPPPTSTHATPRPTTPGPQTTPPGPATPGPVGASAA
PTADSPLTASPPATAPGPSAANVSVAATTATPGTRGTARTPPTDPKTHPHGPADAPPG
SPAPPPPEHRGGPEEFEGAGDGEPPEDDDSATGLAFRTPNPNKPPPARPGPIRPTLPPGI
LGPLAPNTPRPPAQAPAKDMPSGPTPQHIPLFWFLTASPALD,
(SEQ ID No. 9)
MLLVNQSHQGFNKEHTSKMVSAIVLYVLLAAAAHSAFARALRTDPEGVDPD
VRAPRTGRRLMALTEDTSSDSPTSAPEKTPLPVSATAMAPSVDPSAEPTAPATTTPPD
EMATQAATVAVTPEETAVASPPATASVESSPLPAAAAATPGAGHINTSSASAAKTPP
TTPAPTTPPPTSTHATPRPTTPGPQTTPPGPATPGPVGASAAPTADSPLTASPPATAPGP
SAANVSVAATTATPGTRGTARTPPTDPKTHPHGPADAPPGSPAPPPPEHRGGPEEFEG
AGDGEPPEDDDSATGLAFRTPNPNKPPPARPGPIRPTLPPGILGPLAPNTPRPPAQAPA
KDMPSGPTPQHIPLFWFLTASPALDHHHHHH,
(SEQ ID No. 10)
MGWSCIILFLVATATGVHSRALRTDPEGVDPDVRAPRTGRRLMALTEDTSSD
SPTSAPEKTPLPVSATAMAPSVDPSAEPTAPATTTPPDEMATQAATVAVTPEETAVAS
PPATASVESSPLPAAAAATPGAGHTNTSSASAAKTPPTTPAPTTPPPTSTHATPRPTTP
GPQTTPPGPATPGPVGASAAPTADSPLTASPPATAPGPSAANVSVAATTATPGTRGTA
RTPPTDPKTHPHGPADAPPGSPAPPPPEHRGGPEEFEGAGDGEPPEDDDSATGLAFRT
PNPNKPPPARPGPIRPTLPPGILGPLAPNTPRPPAQAPAKDMPSGPTPQHIPLFWFLTAS
PALDHHHHHH,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 1-3 and 8-10, and
d) functional groups for covalently attaching the isolated peptide sequences to a solid support.

28-34. (canceled)

35. The solid support of claim 27, wherein the solid support is not directly linked to an antibody, the solid support further comprises a control region, wherein the control region comprises a control agent, or wherein the solid support comprises at least three regions defining an area of the support including an isolated peptide, and wherein one of the at least three regions include the amino acid sequence as set forth in any one of SEQ ID Nos. 1-3, and another of the at least three regions includes the amino acid sequence as set forth in any one of SEQ ID Nos. 8-10.

36-37. (canceled)

38. A solid support, wherein the support comprises:
a wicking material for directing material flow along a direction of the solid support;
a sample region comprising a first isolated peptide, optionally wherein the first isolated peptide has a concentration of no less than 10 ng/mm² and no greater than 600 ng/mm² based on an area of the sample region, wherein the first isolated peptide is not linked to the solid support, and wherein the first isolated peptide is fused to a heterologous protein; a detectable agent, which detectable agent is optionally a latex bead or a gold particle, a reactive agent, or combinations thereof;
a test region positioned beyond the sample region along the direction of material flow, wherein the test region comprises a second isolated peptide linked to the solid support; and
a control region positioned beyond the test region along the direction of material flow, wherein the control region comprises a control agent, and wherein
each of the first isolated peptide and the second isolated peptide comprise an amino acid sequence set forth as:
(SEQ ID No. 1)
VPTNVSSTTQPQLQTTGRPSHEAPNMTQTGTTDSPTAISLTTPDHTPPM
PSIGLEEEEEEEGAGDGEHLEGGDGTRDTLPQSPGPAFPLAEDVEKDKP
NRPVVPSPDPNNSPARPETSRPKTPPTIIGPLATRPTTRLTSKGRPLVP
TPQHTPLFSFLTASPALDT,
and analogues thereof wherein the first and second isolated peptide sequences comprise one or more of:
a) up to 5 amino acids at the C and/or at the N terminal ends,
b) one, two or three conservative amino acid substitutions,
c) 98% or greater sequence homology to SEQ ID No. 1, and
d) functional groups for covalently attaching the first and/or second isolated peptide sequences to a solid support,
optionally wherein each of the first isolated peptide and the second isolated peptide consists substantially of the amino acid sequence set forth as:
(SEQ ID No. 2)
MLLVNQSHQGFNKEHTSKMVSAIVLYVLLAAAAHSAFAVPTNVSSTTQP
QLQTTGRPSHEAPNMTQTGTTDSPTAISLTTPDHTPPMPSIGLEEEEEE
EGAGDGEHLEGGDGTRDTLPQSPGPAFPLAEDVEKDKPNRPVVPSPDPN
NSPARPETSRPKTPPTIIGPLATRPTTRLTSKGRPLVPTPQHTPLFSFL
TASPALDTHHHHHH,
(SEQ ID No. 3)
MGWSCIILFLVATATGVHSVPTNVSSTTQPQLQTTGRPSHEAPNMTQTG
TTDSPTAISLTTPDHTPPMPSIGLEEEEEEEGAGDGEHLEGGDGTRDTL
PQSPGPAFPLAEDVEKDKPNRPVVPSPDPNNSPARPETSRPKTPPTIIG
PLATRPTTRLTSKGRPLVPTPQHTPLFSFLTASPALDTHHHHHH,
and analogues thereof wherein the first and second isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 2-3, and
d) functional groups for covalently attaching the first and/or second isolated peptide sequences to a solid support

39-41. (canceled)

42. A method for detecting anti-Herpes Simplex Virus antibodies in a biological sample containing antibodies, comprising:
contacting the biological sample with a solid support including an isolated peptide of claim 27 under conditions sufficient to form an immune complex between the isolated peptide and an antibody present in the biological sample, and detecting the presence or absence of the immune complex, wherein the presence of the immune complex indicates anti-HSV antibodies are present in the biological sample.

43-47. (canceled)

48. The method of claim 42, further comprising administering a therapeutic agent to a patient that tests positive for HSV-1 and/or HSV-2 infection, wherein the therapeutic agent is effective at treating the symptoms of a HSV-1 and/or HSV-2 infection, or in treating the underlying viral infection, optionally wherein the therapeutic agent is selected from the group consisting of Acyclovir (Zovirax), Famciclovir (Famvir), Docosanol (Abreva), Denavir and (Penciclovir).

49. (canceled)

50. An isolated peptide having an amino acid sequence set forth as:
(SEQ ID No. 1)
VPTNVSSTTQPQLQTTGRPSHEAPNMTQTGTTDSPTAISLTTPDHTPPMPSIGL
EEEEEEEGAGDGEHLEGGDGTRDTLPQSPGPAFPLAEDVEKDKPNRPVVPSPDPNNS
PARPETSRPKTPPTIIGPLATRPTTRLTSKGRPLVPTPQHTPLFSFLTASPALDT,
(SEQ ID No. 2)
MLLVNQSHQGFNKEHTSKMVSAIVLYVLLAAAAHSAFAVPTNVSSTTQPQL
QTTGRPSHEAPNMTQTGTTDSPTAISLTTPDHTPPMPSIGLEEEEEEEGAGDGEHLEG
GDGTRDTLPQSPGPAFPLAEDVEKDKPNRPVVPSPDPNNSPARPETSRPKTPPTIIGPL
ATRPTTRLTSKGRPLVPTPQHTPLFSFLTASPALDTHHHHHH,
(SEQ ID No. 3)
MGWSCIILFLVATATGVHSVPTNVSSTTQPQLQTTGRPSHEAPNMTQTGTTDS
PTAISLTTPDHTPPMPSIGLEEEEEEEGAGDGEHLEGGDGTRDTLPQSPGPAFPLAEDV
EKDKPNRPVVPSPDPNNSPARPETSRPKTPPTIIGPLATRPTTRLTSKGRPLVPTPQHTP
LFSFLTASPALDTHHHHHH,
(SEQ ID No. 8)
RALRTDPEGVDPDVRAPRTGRRLMALTEDTSSDSPTSAPEKTPLPVSATAMAP
SVDPSAEPTAPATTTPPDEMATQAATVAVTPEETAVASPPATASVESSPLPAAAAATP
GAGHTNTSSASAAKTPPTTPAPTTPPPTSTHATPRPTTPGPQTTPPGPATPGPVGASAA
PTADSPLTASPPATAPGPSAANVSVAATTATPGTRGTARTPPTDPKTHPHGPADAPPG
SPAPPPPEHRGGPEEFEGAGDGEPPEDDDSATGLAFRTPNPNKPPPARPGPIRPTLPPGI
LGPLAPNTPRPPAQAPAKDMPSGPTPQHIPLFWFLTASPALD,
(SEQ ID No. 9)
MLLVNQSHQGFNKEHTSKMVSAIVLYVLLAAAAHSAFARALRTDPEGVDPD
VRAPRTGRRLMALTEDTSSDSPTSAPEKTPLPVSATAMAPSVDPSAEPTAPATTTPPD
EMATQAATVAVTPEETAVASPPATASVESSPLPAAAAATPGAGHTNTSSASAAKTPP
TTPAPTTPPPTSTHATPRPTTPGPQTTPPGPATPGPVGASAAPTADSPLTASPPATAPGP
SAANVSVAATTATPGTRGTARTPPTDPKTHPHGPADAPPGSPAPPPPEHRGGPEEFEG
AGDGEPPEDDDSATGLAFRTPNPNKPPPARPGPIRPTLPPGILGPLAPNTPRPPAQAPA
KDMPSGPTPQHIPLFWFLTASPALDHHHHHH,
(SEQ ID No. 10)
MGWSCIILFLVATATGVHSRALRTDPEGVDPDVRAPRTGRRLMALTEDTSSD
SPTSAPEKTPLPVSATAMAPSVDPSAEPTAPATTTPPDEMATQAATVAVTPEETAVAS
PPATASVESSPLPAAAAATPGAGHTNTSSASAAKTPPTTPAPTTPPPTSTHATPRPTTP
GPQTTPPGPATPGPVGASAAPTADSPLTASPPATAPGPSAANVSVAATTATPGTRGTA
RTPPTDPKTHPHGPADAPPGSPAPPPPEHRGGPEEFEGAGDGEPPEDDDSATGLAFRT
PNPNKPPPARPGPIRPTLPPGILGPLAPNTPRPPAQAPAKDMPSGPTPQHIPLFWFLTAS
PALDHHHHHH,
and analogues thereof wherein the isolated peptide sequences comprise one or more of:
a) up to 5 amino acids at the C and/or at the N terminal ends,
b) one, two or three conservative amino acid substitutions,
c) 98% or greater sequence homology to SEQ ID Nos. 1-3 and 8-10,
d) one or more functional groups for covalently attaching the isolated peptide sequences to a solid support, and
e) a solid support to which the isolated peptide is attached, through the functional group or
f) a particle to which the isolated peptide is attached, either covalently or non-covalently,
optionally further comprising an HSV-1 or HSV-2 antibody bound to the isolated peptide bound to the particle, and optionally wherein the HSV-1 or HSV-2 antibody is bound to a) an isolated peptide bound to a particle, and b) an isolated peptide bound to a solid support.

51-52. (canceled)


SEQUENCE LISTING (13 sequences)

SEQ ID NO: 1 - protein, 166 aa, Herpes Simplex 1
VPTNVSSTTQPQLQTTGRPSHEAPNMTQTGTTDSPTAISLTTPDHTPPMPSIGLEEEEEEEGAGDGEHLEGGDGTRDTLPQSPGPAFPLAEDVEKDKPNRPVVPSPDPNNSPARPETSRPKTPPTIIGPLATRPTTRLTSKGRPLVPTPQHTPLFSFLTASPALDT

SEQ ID NO: 2 - protein, 210 aa, Herpes Simplex 1
MLLVNQSHQGFNKEHTSKMVSAIVLYVLLAAAAHSAFAVPTNVSSTTQPQLQTTGRPSHEAPNMTQTGTTDSPTAISLTTPDHTPPMPSIGLEEEEEEEGAGDGEHLEGGDGTRDTLPQSPGPAFPLAEDVEKDKPNRPVVPSPDPNNSPARPETSRPKTPPTIIGPLATRPTTRLTSKGRPLVPTPQHTPLFSFLTASPALDTHHHHHH

SEQ ID NO: 3 - protein, 191 aa, Herpes Simplex 1
MGWSCIILFLVATATGVHSVPTNVSSTTQPQLQTTGRPSHEAPNMTQTGTTDSPTAISLTTPDHTPPMPSIGLEEEEEEEGAGDGEHLEGGDGTRDTLPQSPGPAFPLAEDVEKDKPNRPVVPSPDPNNSPARPETSRPKTPPTIIGPLATRPTTRLTSKGRPLVPTPQHTPLFSFLTASPALDTHHHHHH

SEQ ID NO: 4 - protein, 166 aa, Herpes Simplex 1; identical to SEQ ID NO: 1

SEQ ID NO: 5 - protein, 172 aa, Herpes Simplex 1
VPTNVSSTTQPQLQTTGRPSHEAPNMTQTGTTDSPTAISLTTPDHTPPMPSIGLEEEEEEEGAGDGEHLEGGDGTRDTLPQSPGPAFPLAEDVEKDKPNRPVVPSPDPNNSPARPETSRPKTPPTIIGPLATRPTTRLTSKGRPLVPTPQHTPLFSFLTASPALDTHHHHHH

SEQ ID NO: 6 - protein, 185 aa, Herpes Simplex 1
MGWSCIILFLVATATGVHSVPTNVSSTTQPQLQTTGRPSHEAPNMTQTGTTDSPTAISLTTPDHTPPMPSIGLEEEEEEEGAGDGEHLEGGDGTRDTLPQSPGPAFPLAEDVEKDKPNRPVVPSPDPNNSPARPETSRPKTPPTIIGPLATRPTTRLTSKGRPLVPTPQHTPLFSFLTASPALDT

SEQ ID NO: 7 - protein, 191 aa, Herpes Simplex 1; identical to SEQ ID NO: 3

SEQ ID NO: 8 - protein, 330 aa, Herpes Simplex 2
RALRTDPEGVDPDVRAPRTGRRLMALTEDTSSDSPTSAPEKTPLPVSATAMAPSVDPSAEPTAPATTTPPDEMATQAATVAVTPEETAVASPPATASVESSPLPAAAAATPGAGHTNTSSASAAKTPPTTPAPTTPPPTSTHATPRPTTPGPQTTPPGPATPGPVGASAAPTADSPLTASPPATAPGPSAANVSVAATTATPGTRGTARTPPTDPKTHPHGPADAPPGSPAPPPPEHRGGPEEFEGAGDGEPPEDDDSATGLAFRTPNPNKPPPARPGPIRPTLPPGILGPLAPNTPRPPAQAPAKDMPSGPTPQHIPLFWFLTASPALD

SEQ ID NO: 9 - protein, 374 aa, Herpes Simplex 2
MLLVNQSHQGFNKEHTSKMVSAIVLYVLLAAAAHSAFARALRTDPEGVDPDVRAPRTGRRLMALTEDTSSDSPTSAPEKTPLPVSATAMAPSVDPSAEPTAPATTTPPDEMATQAATVAVTPEETAVASPPATASVESSPLPAAAAATPGAGHTNTSSASAAKTPPTTPAPTTPPPTSTHATPRPTTPGPQTTPPGPATPGPVGASAAPTADSPLTASPPATAPGPSAANVSVAATTATPGTRGTARTPPTDPKTHPHGPADAPPGSPAPPPPEHRGGPEEFEGAGDGEPPEDDDSATGLAFRTPNPNKPPPARPGPIRPTLPPGILGPLAPNTPRPPAQAPAKDMPSGPTPQHIPLFWFLTASPALDHHHHHH

SEQ ID NO: 10 - protein, 355 aa, Herpes Simplex 2
MGWSCIILFLVATATGVHSRALRTDPEGVDPDVRAPRTGRRLMALTEDTSSDSPTSAPEKTPLPVSATAMAPSVDPSAEPTAPATTTPPDEMATQAATVAVTPEETAVASPPATASVESSPLPAAAAATPGAGHTNTSSASAAKTPPTTPAPTTPPPTSTHATPRPTTPGPQTTPPGPATPGPVGASAAPTADSPLTASPPATAPGPSAANVSVAATTATPGTRGTARTPPTDPKTHPHGPADAPPGSPAPPPPEHRGGPEEFEGAGDGEPPEDDDSATGLAFRTPNPNKPPPARPGPIRPTLPPGILGPLAPNTPRPPAQAPAKDMPSGPTPQHIPLFWFLTASPALDHHHHHH

SEQ ID NO: 11 - protein, 374 aa, Herpes Simplex 2; identical to SEQ ID NO: 9

SEQ ID NO: 12 - protein, 368 aa, Herpes Simplex 2
MLLVNQSHQGFNKEHTSKMVSAIVLYVLLAAAAHSAFARALRTDPEGVDPDVRAPRTGRRLMALTEDTSSDSPTSAPEKTPLPVSATAMAPSVDPSAEPTAPATTTPPDEMATQAATVAVTPEETAVASPPATASVESSPLPAAAAATPGAGHTNTSSASAAKTPPTTPAPTTPPPTSTHATPRPTTPGPQTTPPGPATPGPVGASAAPTADSPLTASPPATAPGPSAANVSVAATTATPGTRGTARTPPTDPKTHPHGPADAPPGSPAPPPPEHRGGPEEFEGAGDGEPPEDDDSATGLAFRTPNPNKPPPARPGPIRPTLPPGILGPLAPNTPRPPAQAPAKDMPSGPTPQHIPLFWFLTASPALD

SEQ ID NO: 13 - protein, 330 aa, Herpes Simplex 2; identical to SEQ ID NO: 8